(19) United States
(12) Patent Application Publication
(10) Pub. No.: US 2021/0315826 A1
(43) Pub. Date: Oct. 14, 2021

(54) NATURAL FLUORESCENT POLYDEDRAL AMINO ACID CRYSTALS FOR EFFICIENT ENTRAPMENT AND SYSTEMIC DELIVERY OF HYDROPHOBIC SMALL MOLECULES

(71) Applicant: CORNELL UNIVERSITY, Ithaca, NY (US)

(72) Inventors: Alireza ABBASPOURRAD, Ithaca, NY (US); Raheleh RAVANFAR, Ithaca, NY (US)

(21) Appl. No.: 17/262,494

(22) PCT Filed: Jul. 23, 2019
(86) PCT No.: PCT/US19/43030

§ 371 Date: Jan. 22, 2021

Related U.S. Application Data
(60) Provisional application No. 62/702,058, filed on Jul. 23, 2018.

(51) Int. Cl. A61K 9/48; A61K 31/704; A61K 8/11; A61K 8/73; A61K 8/49; A61K 8/60; G01N 33/52; G01N 33/50; G01N 21/64; A61K 45/06

(52) U.S. Cl. CPC A61K 9/4816; A61K 31/704; A61K 9/4866; A61K 8/11; A61K 8/735; A61K 45/06; A61K 8/602; G01N 33/52; G01N 33/5005; G01N 21/6458; A61K 8/4946

(57) ABSTRACT

The present invention relates to an encapsulated product that includes one or more amino acids, where the one or more amino acids are in the form of a crystal with one or more hydrophobic domains and one or more hydrophobic agents entrapped within the hydrophobic domains of the crystal of the one or more amino acids, the crystal having a hydrophilic exterior. Pharmaceutical and cosmetic compositions comprising the encapsulated product, methods of therapeutically treating a subject with the encapsulated product, as well as methods of in vitro imaging and methods of preparing an encapsulated product are also disclosed.

This application claims the priority benefit of U.S. Provisional Patent Application Ser. No. 62/702,058, filed Jul. 23, 2018, which is hereby incorporated by reference in its entirety.

### FIELD

The present application relates to an encapsulated product, a pharmaceutical or cosmetic composition including the encapsulated product described herein, methods of therapeutically treating a subject, methods of in vitro imaging, and methods of preparing an encapsulated product.

### BACKGROUND

The clinical use of various potent, hydrophobic molecules is often hampered by their poor water solubility (Liu et al., “PEGylated Nanographene Oxide for Delivery of Water-Insoluble Cancer Drugs,” J. Am. Chem. Soc. 130(33):10876-10877 (2008)). Low water solubility results in poor absorption as well as low biodistribution and bioavailability of hydrophobic therapeutics upon oral administration (Lipinski et al., “Experimental and Computational Approaches to Estimate Solubility and Permeability in Drug Discovery and Development Settings,” Adv. Drug Deliv. Rev. 46(1-3):3-26 (2001)). Moreover, low water solubility causes drug aggregation upon intravenous administration, which is associated with local toxicity and lowered systemic bioavailability (Fernandez et al., “N-Succinyl-(beta-alanyl-L-leucyl-L-alanyl-L-leucyl)doxorubicin: An Extracellularly Tumor-Activated Prodrug Devoid of Intravenous Acute Toxicity,” J. Med. Chem. 44(22):3750-3753 (2001) and Allen et al., “Drug Delivery Systems: Entering the Mainstream,” Science 303(5665):1818-1822 (2004). For example, doxorubicin (DOX) is a widely used hydrophobic anticancer drug with excellent anti-neoplastic activity against a multitude of human cancers (Fritze et al., “Remote Loading of Doxorubicin into Liposomes Driven by a Transmembrane Phosphate Gradient,” Biochim. Biophys. Acta. 1758(10):1633-40 (2006)). However, its clinical use is hindered by acute side effects, such as vomiting, bone marrow suppression, and drug-induced irreversible cardiotoxicity (Wang et al., “Doxorubicin Induces Apoptosis in Normal and Tumor Cells via Distinctly Different Mechanisms. Intermediacy of H(2)O(2)- and p53-Dependent Pathways,” J. Biol. Chem. 279(24):25535-25543 (2004)). Most of these side effects are due to the poor water solubility of DOX (Torchilin V P, “Targeted Polymeric Micelles for Delivery of Poorly Soluble Drugs,” Cell Mol. Life Sci. 61(19-20):2549-2559 (2004)).

These challenges have driven the development of drug-delivery systems to increase the efficacy of hydrophobic therapeutics through improved pharmacokinetics and biodistribution (Kim et al., “Entrapment of Hydrophobic Drugs in Nanoparticle Monolayers with Efficient Release into Cancer Cells,” J. Am. Chem. Soc. 131(4):1360-1361 (2009)). A wide variety of scaffolds, such as liposomes (Allen et al., “Liposomal Drug Delivery Systems: From Concept to Clinical Applications,” Adv. Drug Deliv. Rev. 65(1):36-48 (2013)) and stimuli-responsive polymeric particles (Hoffman A S, “Stimuli-Responsive Polymers: Biomedical Applications and Challenges for Clinical Translation,” Adv. Drug Deliv. Rev. 65(1):10-16 (2013) and Ravanfar et al., “Controlling the Release from Enzyme-Responsive Microcapsules with a Smart Natural Shell,” ACS Appl. Mater. Interfaces 10(6):6046-6053 (2018)), have been explored, either covalently or noncovalently conjugating hydrophobic drugs with these systems (Kim et al., “Entrapment of Hydrophobic Drugs in Nanoparticle Monolayers with Efficient Release into Cancer Cells,” J. Am. Chem. Soc. 131(4):1360-1361 (2009)). Despite significant advances in the development of such drug carriers, there remain a few problems that have resulted in therapeutic failure, including the lack of site-specificity (Zhu et al., “Drug Delivery: Tumor-Specific Self-Degradable Nanogels as Potential Carriers for Systemic Delivery of Anticancer Proteins,” Adv. Funct. Mater. 28(17):1707371 (2018)), low biocompatibility (Maiti et al., “Redox-Responsive Core-Cross-Linked Block Copolymer Micelles for Overcoming Multidrug Resistance in Cancer Cells,” ACS Appl. Mater. Interfaces 10(6):5318-5330 (2018)), and inefficient drug entrapment within the carriers (Miatmoko et al., “Evaluation of Cisplatin-Loaded Polymeric Micelles and Hybrid Nanoparticles Containing Poly(Ethylene Oxide)-Block-Poly(Methacrylic Acid) on Tumor Delivery,” Pharmacology and Pharmacy 7(1):1-8 (2016)). Moreover, covalent attachment in some cases requires chemical modification, which can reduce the efficiency of drug release or incomplete intracellular processing of a prodrug compound.[13] These strategies also involve additional complexities associated with mass production difficulties and cost. Thus, the fabrication of biocompatible platforms that can overcome these limitations remains an important yet unmet need.

The present application is directed to overcoming these and other deficiencies in the art.

### SUMMARY

One aspect of the present application relates to an encapsulated product comprising (i) one or more amino acids, where the one or more amino acids are in the form of a crystal with one or more hydrophobic domains and (ii) one or more hydrophobic agents entrapped within the hydrophobic domains of the crystal of the one or more amino acids, the crystal having a hydrophilic exterior.

Another aspect of the present application relates to a pharmaceutical or cosmetic composition comprising a pharmaceutically or cosmetically acceptable carrier and the encapsulated product as described herein.

A further aspect of the present application relates to a method of therapeutically treating a subject with one or more hydrophobic agents. This method involves selecting a subject in need of therapeutic treatment and administering the encapsulated product or pharmaceutical or cosmetic composition described herein to the selected subject.

Yet another aspect of the present application relates to a method of in vitro imaging. This method involves contacting the in vitro cell culture system with the encapsulated product or pharmaceutical or cosmetic composition described herein and imaging the contacted cell culture system.

Another aspect of the present application relates to a method of preparing an encapsulated product comprising entrapped hydrophobic agents. This method involves mixing one or more hydrophobic agents with one or more amino acids to produce a mixture and forming crystals of the one or more amino acids entrapping the one or more hydrophobic agents, where the crystals have a hydrophilic exterior.

The results described herein demonstrate that L-histidine (L-His) crystals can function as efficient vehicles to entrap hydrophobic free drugs, such as doxorubicin (DOX), as well as other hydrophobic small molecules, including Nile red, β-carotene, and pyrene (FIGS. 1A-1B). The noncovalent inclusion of such hydrophobic molecules inside the hydrophobic domains within the interior of the polymorph A crystal structure of L-His suggests the capability for efficient drug transport and release, avoiding prodrug processing issues. As an essential amino acid, L-His crystals also have the advantage of being biocompatible and feature the ability to load a large quantity of hydrophobic molecules. Furthermore, the examples presented herein demonstrate the natural fluorescent properties of L-His crystals, which suggests their potential as traceable compounds inside biological systems.

As described herein, L-His crystals can be chemically modified at the surface to provide preferential biological targeting to the desired site of action (FIG. 1C). By covalently cross-linking hyaluronic acid (HA) to the surface of L-His crystals (HA-His crystals), applicant demonstrates that hyaluronidase (HAase) hydrolyzes the HA on the HA-His crystals, allowing the L-His crystals to dissolve in an aqueous matrix and release encapsulated small molecules, such as DOX, to a desired site. This scaffold provides highly efficient noncovalent inclusion of hydrophobic molecules or active drugs with excellent biocompatibility and efficient bioresponsive drug release. Moreover, the HA-His crystals are potentially site-specific, making them excellent candidates for targeting CD44-receptors overexpressed on tumors, and thus enhancing the permeability of anticancer drugs.

### BRIEF DESCRIPTION OF THE DRAWINGS

FIGS. 1A-1C illustrate the preparation of L-Histidine (L-His) crystals. FIG. 1A is a schematic representation for the preparation of L-His crystals loaded with DOX molecules. FIG. 1B show confocal laser scanning microscopy (CLSM) images of (i) L-His crystal emitted with green color and (ii) DOX with red color in L-His crystal. FIG. 1C is a schematic representation for the preparation of L-His crystals surface-modified with tumor-specific HA for the targeted delivery of hydrophobic DOX molecules.

FIGS. 2A-2D illustrate the X-Ray Diffraction (XRD) pattern and arrangement of L-His molecules within L-His crystals. FIG. 2A show fluorescence microscopy images of L-His crystals. FIG. 2B shows the simulated and experimental XRD patterns of pure L-His crystals. FIG. 2C is a Ball and stick representation of four L-His molecules arranged in the polymorph A with the orthorhombic space group P212121, showing the hydrophobic domain surrounded by imidazole rings of the L-His molecules. FIG. 2D is a ball and stick representation for the unit cell of crystals formed after loading the small molecules, showing two L-His molecules with monoclinic space group P21.

FIGS. 3A-3D show (i) digital images, (ii) optical microscopy images, (iii) scanning electron microscopy images (SEM), and (iv) CLSM images of various L-His crystals. FIG. 3A shows images of pure L-His crystals; the green color in (iv) represents the pure L-His crystals. FIG. 3B shows images of β-carotene-entrapped L-His crystals; the green color in (iv) represents the L-His crystals and the orange color represents β-carotene. FIG. 3C shows images of Nile red-entrapped L-His crystals; the green color in (iv) represents the L-His crystals and the red color represents Nile red. FIG. 4D shows images of pyrene-entrapped L-His crystals; the green color in (iv) represents the L-His crystals and the blue color represents pyrene. First column (i): digital images; second column (ii): optical microscopy images; third column (iii): SEM images; fourth column (iv): CLSM images. Scale bars: 100 m.

FIGS. 4A-4G show CLSM data of L-His crystals loaded with Nile Red, pyrene, and β-carotene, respectively. FIG. 4A shows CLSM imaging data collected at different dimensions of the L-His crystals, confirming the localization of the hydrophobic Nile red inside the L-His crystals. FIG. 4B shows an ortho demonstration of L-His crystals with entrapped Nile red. FIG. 4C shows an ortho demonstration of L-His crystals with entrapped pyrene. FIG. 4D shows CLSM imaging data of L-His crystals with entrapped β-carotene in 2D. FIGS. 4D-4F show CLSM imaging data of L-His crystals with entrapped β-carotene in 2.5D, with intensity on the Z-axis. FIG. 4G shows L-His crystals with entrapped Nile red in 2D. FIGS. 4H-4I show L-His crystals with entrapped Nile red in 2.5D, confirming the localization of the hydrophobic small molecules inside the L-His crystals. The green and blue colors represent the L-His crystals, and the orange and red colors represent the β-carotene, pyrene, and Nile red, respectively. Scale bars: 100 m.

FIGS. 5A-5G shows XRD patterns and SEM images of L-His crystals loaded with β-carotene, Nile red, pyrene, and DOX, respectively. FIG. 5A shows the XRD patterns of the L-His crystals with entrapped small molecules (green lines) in comparison with the L-His crystals (red lines), the small molecules (black lines), a mixture of L-His and the small molecules (blue lines), and surface-modified L-His crystals with entrapped small molecules (pink lines) for i) 3-carotene, ii) Nile red, iii) pyrene, and iv) DOX. FIG. 5B shows SEM images of the L-His crystals before surface modification; FIG. 5C is a magnification of FIG. 5B. FIG. 5D shows the L-His crystals after chemical surface modification through disulfide bonds with HA; FIG. 5E is a magnification of FIG. 5D, with the inset showing a further-magnified image. FIG. 5F shows the L-His crystals after surface modification through manual mixing of the crystals with HA solution; FIG. 5G is a magnification of FIG. 5F.

FIGS. 6A-6C illustrates the process used for chemically modifying the surface of L-His crystals. FIG. 6A is a schematic illustration for the synthesis of (i) SH-HA and (ii) SH-HME. FIG. 6B shows the fourier transform infrared (FTIR) spectra of HA and SH-HA, showing a significant decrease of the peak at 1610-1620 cm-1 associated with the HA carboxyl groups, confirming the formation of SH-HA. FIG. 6C illustrates the formation of disulfide bonds between i and ii, and formation of iii.

FIGS. 7A-7C illustrate the enzymatic degradation of HA-His crystals in the presence of 1 or 10 U/mL HAase at 37° C. FIG. 7A is a schematic illustration of HA-His crystals that can be degraded by HAase, and digital images of the HA-His crystals after four hours without the presence of HAase (control, i) and in the presence of HAase (ii). FIG. 7B is a graph showing the cumulative release of DOX from HA-His crystals. FIG. 7C is a schematic illustration of the enhanced delivery of hydrophobic chemotherapeutics by the HA-His crystals for cancer therapy: (i) HA-His crystals accumulate in the tumor; (ii) HA-His crystals are internalized by the CD44 receptors on the tumor cells; iii) HAase leads to the degradation of HA on the crystal surface, dissolving the crystals; and iv) release of the hydrophobic chemotherapeutics over time to cause the tumor cell death.

FIGS. 8A-8C illustrate the fluorescence of amino acid crystals. FIG. 8A is a schematic representation for the fluorescence of amino acids in the crystalline solid state in comparison with the non-fluorescence aqueous solution of amino acids. FIG. 8B is a schematic representation of Jablonski diagram for fluorescence. FIG. 8C shows CLSM images of the amino acid crystals: i) L-histidine, ii) L-glutamine, iii) L-isoleucine, iv) L-asparagine, v) L-valine, vi) L-threonine, and vii) L-methionine, showing their bright fluorescence emission in a wide range, including blue (414-459 nm, first column), green (500-559, second column), and red wavelengths (587-673, third column). The fourth column shows the bright field images of the amino acid crystals.

FIGS. 9A-9B illustrate the fluorescence of L-His (FIG. 9A) and L-isoleucine (FIG. 9B) crystals. Panel (i) shows the confocal lambda scan of crystals excited at 405 nm, 488 nm, and 561 nm. The numbers on each image correspond to the emission wavelengths. Panel (ii) shows the fluorescent life-time of crystals at room temperature. The red lines represent the biexponential fits to the experimental data points (black lines). Panel (iii) shows the residuals of fluorescent life-time of crystals fitted to a bi-exponential decay curve.

FIGS. 10A-10D show the structure of L-histidine (FIG. 10A), L-glutamine (FIG. 10B), L-isoleucine (FIG. 10C), and L-asparagine (FIG. 10D). Panel (i) shows the crystalline structure of amino acids with their intermolecular hydrogen bonds as determined by X-ray crystallography. Panel (ii) shows the XRD spectra of the crystals. Panel (iii) shows SEM images of the crystals.

FIG. 11 are images showing the confocal lambda scan of L-glutamine crystals excited at 405 nm, 488 nm, and 561 nm.

FIG. 12 are images showing the confocal lambda scan of L-asparagine crystals excited at 405 nm, 488 nm, and 561 nm.

FIG. 13 are images showing the confocal lambda scan of L-valine crystals excited at 405 nm, 488 nm, and 561 nm.

FIG. 14 are images showing the confocal lambda scan of L-threonine crystals excited at 405 nm, 488 nm, and 561 nm.

FIG. 15 are images showing the confocal lambda scan of L-methionine crystals excited at 405 nm, 488 nm, and 561 nm.

FIGS. 16A-16G are emission spectra of amino acid crystals: L-histidine (FIG. 16A), L-glutamine (FIG. 16B), L-isoleucine (FIG. 16C), L-asparagine (FIG. 16D), L-valine (FIG. 16E), L-threonine (FIG. 16F), and L-methionine (FIG. 16G).

FIGS. 17A-17E illustrate the fluorescent life-time of amino acid crystals at room temperature: L-glutamine (FIG. 17A), L-asparagine (FIG. 17B), L-threonine (FIG. 17C), L-methionine (FIG. 17D), and L-valine (FIG. 17E). The red lines represent the biexponential fits to the experimental data points (black lines).

FIGS. 18A-18E show the residuals of fluorescent life-time of amino acid crystals fitted to a bi-exponential decay curve: L-glutamine (FIG. 18A), L-asparagine (FIG. 18B), L-threonine (FIG. 18C), L-methionine (FIG. 18D), and L-valine (FIG. 18E).

FIG. 19 shows FLTM data of a histidine crystal. The image is color-coded by the weighed mean lifetime, showing that the value varies across the crystal surface. The histogram shows the distribution of lifetimes of all the pixels measured.

FIGS. 20A-20C shows the crystalline structure of amino acids with their intermolecular hydrogen bonds: L-valine (FIG. 20A), L-threonine (FIG. 20B), and L-methionine (FIG. 20C).

FIGS. 21A-21B show the FTIR spectra of the L-histidine and deuterated L-histidine crystals in the range of 400-4000 cm−1 (FIG. 21A). FIG. 21B is a close-up view of FIG. 21A in the range of 400-1400 cm⁻¹.

FIGS. 22A-22C show the XRD spectra for the crystals of L-valine (FIG. 22A), L-threonine (FIG. 22B), and L-methionine (FIG. 22C).

FIGS. 23A-23C show SEM images of amino acid crystals: L-valine (FIG. 23A), L-threonine (FIG. 23B), and L-methionine (FIG. 23C).

### DETAILED DESCRIPTION

In this specification and the appended claims, the singular forms “a”, “an”, and “the” include plural references unless the context clearly dictates otherwise.

The terms “comprising”, “comprises”, and “comprised of”, as used herein, are synonymous with “including”, “includes” or “containing”, “contains”, and are inclusive or open-ended and do not exclude additional, non-recited members, elements, or method steps.

The recitation of numerical ranges by endpoints includes all numbers and fractions subsumed within the respective ranges, as well as the recited endpoints.

The terms “encapsulated” or “loaded” and their derivatives are used interchangeably. According to the present application, an “encapsulated product” refers to an amino acid crystal having a hydrophilic agent (e.g., a drug or a therapeutic agent) located in the crystal.

One aspect of the present application relates to an encapsulated product comprising (i) one or more amino acids, where the one or more amino acids are in the form of a crystal with one or more hydrophobic domains and (ii) one or more hydrophobic agents entrapped within the hydrophobic domains of the crystal of the one or more amino acids, the crystal having a hydrophilic exterior.

The one or more amino acids may be aromatic, non-aromatic, or combinations thereof.

Suitable aromatic amino acids include, without limitation, any one or more of histidine, phenylalanine, tyrosine, tryptophan, and derivatives thereof.

Suitable non-aromatic amino acids include, without limitation, glutamine, isoleucine, asparagine, valine, threonine, methionine, and derivatives thereof.

Any known or hereinafter developed histidine derivatives, phenylalanine derivatives, tyrosine derivatives, tryptophan derivatives, glutamine derivatives, isoleucine derivatives, asparagine derivatives, valine derivatives, threonine derivatives, or methionine derivatives can be used in the encapsulated product of the present application. Examples of amino acid derivatives include amino acids with one or more substitutions. In some embodiments, the one or more amino acids is a tryptophan derivative, e.g., 4-cyanotryptophan (Hilaire et al., “Blue Fluorescent Amino Acid for Biological Spectroscopy and Microscopy,” PNAS 114(23):6005-6009 (2017), which is hereby incorporated by reference in its entirety).

In some embodiments, the encapsulated product includes all aromatic amino acids, all non-aromatic amino acids, or a mixture of aromatic and non-aromatic amino acids. For example, when the encapsulated product includes all aromatic amino acids, the one or more amino acids may be histidine. In another example, when the encapsulated product includes all non-aromatic amino acids, the one or more amino acids may be isoleucine.

In some embodiments, the encapsulated product comprises a crystal of one amino acid (i.e., the one or more amino acids are all the same amino acid). In accordance with these embodiments, the one or more hydrophobic agents may be entrapped in a crystal of histidine, phenylalanine, tyrosine, glutamine, isoleucine, asparagine, valine, threonine, methionine, or derivatives thereof.

In some embodiments, the encapsulated product comprise a crystal of at least two amino acids (i.e., the one or more amino acids include two or more amino acids). In accordance with these embodiments, the one or more hydrophobic agents may be entrapped in a cocrystal of at least two amino acids selected from the group consisting of histidine, phenylalanine, tyrosine, glutamine, isoleucine, asparagine, valine, threonine, methionine, or derivatives thereof. As used herein, the term “at least two amino acids” refers to 2, 3, 4, 5, 6, 7, 9, 10, or more amino acids or derivatives thereof.

The one or more amino acids may be L-amino acids, D-amino acids, or combinations thereof. For example, the one or more amino acids may include only L-amino acids, only D-amino acids, or a mixture of L-amino acids and D-amino acids.

Suitable L-amino acids include, without limitation, L-histidine, L-phenylalanine, L-tyrosine, L-tryptophan, L-glutamine, L-isoleucine, L-asparagine, L-valine, L-threonine, L-methionine, and derivatives thereof. In some embodiments, the one or more amino acids is L-histidine.

Suitable D-amino acids may be selected from the group consisting of D-histidine, D-phenylalanine, D-tyrosine, D-tryptophan, D-glutamine, D-isoleucine, D-asparagine, D-valine, D-threonine, D-methionine, and derivatives thereof. In some embodiments the one or more amino acids is D-histidine or a combination of L-histidine and D-histidine, where at least 10%, 15%, 20%, 25%, 30%, 35%, 40%, 45%, 50%, 55%, 60%, 65%, 70%, 75%, 80%, 85%, 90%, 95%, 99%, or more is L-histidine.

In some embodiments, the one or more amino acids are monomers, dimers, trimers, or combinations thereof. As used herein, the term “monomer” refers to a single unit (e.g., a single amino acid), which can be linked with the same unit or other units to form an oligomer (e.g., a dimer or trimer). The term “dimer” refers to an oligomer consisting of two monomers joined together. The dimers may be homodimers or heterodimers. The term “trimer” refers to a polymer consisting of three monomers joined together. The trimers may be homotrimers or heterotrimers.

The one or more hydrophobic agents may be selected from the group consisting of vitamins, carotenoids, antioxidants, drugs, imaging agents, and combinations thereof.

In some embodiments, the one or more hydrophobic agents is a vitamin selected from the group consisting of vitamin A, vitamin D, vitamin E, vitamin K, and combinations thereof.

As described herein, vitamin A is required for the formation of rhodopsin, a photoreceptor pigment in the retina and helps maintain epithelial tissues (see, e.g., Porter, Robert S, and Justin L. Kaplan. The Merck Manual of Diagnosis and Therapy. 2018, which is hereby incorporated by reference in its entirety).

As described herein, vitamin D has two main forms: D₂ (ergocalciferol) and D₃ (cholecalciferol). Vitamin D and related analogs may be used to treat psoriasis, hypoparathyroidism, and renal osteodystrophy (see, e.g., Porter, Robert S, and Justin L. Kaplan. The Merck Manual of Diagnosis and Therapy. 2018, which is hereby incorporated by reference in its entirety). In some embodiments, the vitamin D is D₃.

As described herein, vitamin E is a group of compounds (including tocopherols and tocotrienols) that have similar biologic activities include, e.g., α-tocopherol, β-tocopherol, γ-tocopherol, and S-tocopherol (see, e.g., Porter, Robert S, and Justin L. Kaplan. The Merck Manual of Diagnosis and Therapy. 2018, which is hereby incorporated by reference in its entirety). These compounds act as antioxidants, which prevent lipid peroxidation of polyunsaturated fatty acids in cellular membranes. In some embodiments, the vitamin E is selected from the group consisting of

As described herein, vitamin K controls the formation of coagulation factors II (prothrombin), VII, IX, and X in the liver (see, e.g., Porter, Robert S, and Justin L. Kaplan. The Merc kManual of Diagnosis and Therapy. 2018, which is hereby incorporated by reference in its entirety). Other coagulation factors dependent on vitamin K are protein C, protein S, and protein Z; proteins C and S are anticoagulants. Metabolic pathways conserve vitamin K. Once vitamin K has participated in formation of coagulation factors, the reaction product, vitamin K epoxide, is enzymatically converted to the active form, vitamin K hydroquinone.

As used herein, the term “carotenoid” refers to a class of hydrocarbons having a conjugated polyene carbon skeleton formally derived from isoprene. The term “carotenoid” may include both carotenes and xanthophylls. A “carotene” refers to a hydrocarbon carotenoid (e.g., phytoene, β-carotene, lycopene). The term “xanthophyll” refers to a C₄₀ carotenoid that contains one or more oxygen atoms in the form of hydroxy-, methoxy-, oxo-, epoxy-, carboxy-, or aldehydic functional groups (e.g., β-cryptoxanthin, neoxanthin, violaxanthin).

In some embodiments, the one or more hydrophobic agents is a carotenoid selected from the group consisting of β-carotene, α-carotene, β-cryptoxanthin, lycopene, lutein, zeaxanthin, and combinations thereof.

β-carotene, α-carotene, and β-cryptoxanthin are provitamin A carotenoids, whereas lycopene, lutein, and zeaxanthin have no vitamin A activity and are referred to as non-provitamin A carotenoids (see, e.g., “β-Carotene and Other Carotenoids,” Dietary Reference Intakes for Vitamin C, Vitamin E, Selenium, and Carotenoids. Institute of Medicine (US) Panel on Dietary Antioxidants and Related Compounds. Washington (DC): National Academies Press (2000), which is hereby incorporated by reference in its entirety). Lycopene functions as an antioxidant (Müller et al., “Lycopene and Its Antioxidant Role in the Prevention of Cardiovascular Diseases-A Critical Review,” Crit. Rev. Food Sci. Nutr. 56(110:1868-1879 (2017), which is hereby incorporated by reference in its entirety). Lutein and zeaxanthin are selectively taken up into the macula of the eye, where they absorb up to 90% of blue light and help maintain optimal visual function (Mares J., “Lutein and Zeaxanthin Isomers in Eye Health and Disease.” Annu. Rev. Nutr. 36:571-602 (2016), which is hereby incorporated by reference in its entirety).

The one or more hydrophobic agents may be an antioxidant selected from the group consisting of melatonin, vitamin A, and vitamin E.

Melatonin is a hormone involved in sleep regulatory activity, and a tryptophan-derived neurotransmitter, which inhibits the synthesis and secretion of other neurotransmitters such as dopamine and GABA. Melatonin is synthesized from serotonin intermediate in the pineal gland and the retina where the enzyme 5-hydroxyindole-O-methyltransferase, that catalyzes the last step of synthesis, is found. This hormone binds to and activates melatonin receptors and is involved in regulating the sleep and wake cycles. In addition, melatonin possesses antioxidative and immunoregulatory properties via regulating other neurotransmitters.

Vitamin A and vitamin E are described in more detail above.

The one or more hydrophobic agents may be a drug. In some embodiments, the drug is a chemotherapeutic agent. As used herein, the term “chemotherapeutic agent” refers to a chemical compound that is (e.g., a drug) or becomes (e.g., a prodrug), for example, selectively destructive or selectively toxic to the causative agent of a disease, such as malignant cells and tissues, viruses, bacteria, or other microorganism.

Suitable chemotherapeutic agents include, without limitation, Abarelix, aldesleukin, Aldesleukin, Alemtuzumab, Alitretinoin, Allopurinol, Altretamine, Amifostine, anastrozole, arsenic trioxide, asparaginase, azacitidine, BCG Live, Bevacuzimab, Avastina, Fluorouracil, bexarotene, bleomycin, bortezomib, busulfan, Calusterone, capecitabine, camptothecin, carboplatin, carmustine, Celecoxib, Cetuximab, chlorambucil, cisplatin, cladribine, clofarabine, Cyclophosphamide, Cytarabine, Dactinomycin, Darbepoetin alfa, daunorubicin, denileukin, Dexrazoxane, Docetaxel, Doxorubicin (neutral), Doxorubicin hydrochloride, Dromostanolone propionate, Epirubicin, Epoetin alfa, Erlotinib, Estramustine, Etoposide Phosphate, Etoposide, Exemestane, Filgrastim, floxuridine fludarabine, Fulvestrant, Gefitinib, gemcitabine, Gemtuzumab goserelin acetate, histrelin acetate, hydroxyurea, Ibritumomab, idarubicin, ifosfamide, imatinib mesylate, Interferon Alfa-2a, interferon alfa-2b, irinotecan, Lenalidomide, letrozole, leucovorin, leuprolide acetate, levamisole, Lomustine, Megestrol Acetate, Melphalan, Mercaptopurine, 6-MP, Mesna, Methotrexate, Methoxsalen, Mitomycin C, Mitotane, Mitoxantrone, Nandrolone, Nelarabine Verluma, Oprelvekin, Oxaliplatin, Paclitaxel, Palifermin, Pamidronate, pegademase, Pegaspargase, Pegfilgrastim, disodium Pemetrexed, Pentostatin, Pipobroman, Plicamycin, Porfimer Sodium, Procarbazine, Quinacrine, Rasburicase, Rituximab, Sargramostim, Sorafenib, Streptozocin, sunitinib malate, Talc, Tamoxifen, Temozolomide, Teniposide, VM-26, Testolactone, Thioguanine, 6-TG, thiotepa, topotecan, toremifene, tositumomab, trastuzumab, Tretinoin, ATRA, uracil mustard, valrubicin, vinblastine, vincristine, vinorelbine, Zoledron Zoledronic acid, adriamycin, actinomycin D, colchicine, emetine, trimetrexate, metoprine, cyclosporine, amphotericin, 5 fluorouracil, and metronidazole.

In some embodiments, the one or more hydrophobic agents is a drug selected from the group consisting of anticancer agents and antimicrobial agents.

As used herein, the terms “cancer” and “cancerous” refer to or describe the physiological condition in which a population of cells are characterized unregulated cell growth. Examples of cancer include, but are not limited to, carcinoma, sarcoma, melanoma, leukemia, lymphoma, and combinations thereof (mixed-type cancer). A “carcinoma” is a cancer originating from epithelial cells of the skin or the lining of the internal organs. A “sarcoma” is a tumor derived from mesenchymal cells, usually those constituting various connective tissue cell types, including fibroblasts, osteoblasts, endothelial cell precursors, and chondrocytes. A “melanoma” is a tumor arising from melanocytes, the pigmented cells of the skin and iris. A “leukemia” is a malignancy of any of a variety of hematopoietic stem cell types, including the lineages leading to lymphocytes and granulocytes, in which the tumor cells are nonpigmented and dispersed throughout the circulation. A “lymphoma” is a solid tumor of the lymphoid cells. More particular examples of such cancers include, e.g., acinar cell carcinoma, adenocarcinoma (ductal adenocarcinoma), adenosquamous carcinoma, anaplastic carcinoma, cystadenocarcinoma, duct-cell carcinoma (ductal adrenocarcinoma), giant-cell carcinoma (osteoclastoid type), mixed-cell carcinoma, mucinous (colloid) carcinoma, mucinous cystadenocarcinoma, papillary adenocarcinoma, pleomorphic giant-cell carcinoma, serous cystadenocarcinoma, and small-cell (oat-cell) carcinoma. As used herein, cancers are named according to the organ in which they originate.

The term “anticancer agent” refers to a therapeutic agent (e.g., chemotherapeutic coumpounds and/or molecular therapeutic compounds) used in the treatment of a cancer. In some embodiments, when the one or more hydrophobic agents is an anticancer agent, the anticancer agent is selected from the group consisting of doxorubicin HCl (Dox), paclitaxel (PTX), 5-fluorouracil, camptothecin, cisplatin, metronidazole, melphalan, docetaxel, and combinations thereof.

Doxorubicin HCl is the hydrochloride salt of doxorubicin, an anthracycline antibiotic with antineoplastic activity. Doxorubicin intercalates between base pairs in the DNA helix, thereby preventing DNA replication and ultimately inhibiting protein synthesis. Additionally, doxorubicin inhibits topoisomerase II which results in an increased and stabilized cleavable enzyme-DNA linked complex during DNA replication and subsequently prevents the ligation of the nucleotide strand after double-strand breakage. Doxorubicin also forms oxygen free radicals resulting in cytotoxicity secondary to lipid peroxidation of cell membrane lipids; the formation of oxygen free radicals also contributes to the toxicity of the anthracycline antibiotics, namely the cardiac and cutaneous vascular effects.

Paclitaxel is a compound extracted from the Pacific yew tree Taxus brevifolia with antineoplastic activity. Paclitaxel binds to tubulin and inhibits the disassembly of microtubules, thereby resulting in the inhibition of cell division. This agent also induces apoptosis by binding to and blocking the function of the apoptosis inhibitor protein Bcl-2 (B-cell Leukemia 2)

5-fluoruracil is an antimetabolite fluoropyrimidine analog of the nucleoside pyrimidine with antineoplastic activity. In vivo, 5-fluoruracil is converted to the active metabolite 5-fluoroxyuridine monophosphate (F-UMP); replacing uracil, F-UMP incorporates into RNA and inhibits RNA processing, thereby inhibiting cell growth. Another active metabolite, 5-5-fluoro-2′-deoxyuridine-5′-O-monophosphate (F-dUMP), inhibits thymidylate synthase, resulting in the depletion of thymidine triphosphate (TTP), one of the four nucleotide triphosphates used in the in vivo synthesis of DNA. Other fluorouracil metabolites incorporate into both RNA and DNA; incorporation into RNA results in major effects on both RNA processing and functions.

Camptothecin is an alkaloid isolated from the Chinese tree Camptotheca acuminata, with antineoplastic activity. During the S phase of the cell cycle, camptothecin selectively stabilizes topoisomerase I-DNA covalent complexes, thereby inhibiting religation of topoisomerase I-mediated single-strand DNA breaks and producing potentially lethal double-strand DNA breaks when encountered by the DNA replication machinery.

Cisplatin is an alkylating-like inorganic platinum agent (cis-diamminedichloroplatinum) with antineoplastic activity. Cisplatin forms highly reactive, charged, platinum complexes which bind to nucleophilic groups such as GC-rich sites in DNA inducing intrastrand and interstrand DNA cross-links, as well as DNA-protein cross-links. These cross-links result in apoptosis and cell growth inhibition.

Metronidazole is a synthetic nitroimidazole derivative with antiprotozoal and antibacterial activities. Un-ionized metronidazole is readily taken up by obligate anaerobic organisms and is subsequently reduced by low-redox potential electron-transport proteins to an active, intermediate product. Reduced metronidazole causes DNA strand breaks, thereby inhibiting DNA synthesis and bacterial cell growth.

Melphalan is a phenylalanine derivative of nitrogen mustard with antineoplastic activity. Mel A phenylalanine derivative of nitrogen mustard with antineoplastic activity. Melphalan alkylates DNA at the N7 position of guanine and induces DNA inter-strand cross-linkages, resulting in the inhibition of DNA and RNA synthesis and cytotoxicity against both dividing and non-dividing tumor cells. phalan alkylates DNA at the N7 position of guanine and induces DNA inter-strand cross-linkages, resulting in the inhibition of DNA and RNA synthesis and cytotoxicity against both dividing and non-dividing tumor cells.

Docetaxel is a semi-synthetic, second-generation taxane derived from a compound found in the European yew tree, Taxus baccata. Docetaxel displays potent and broad antineoplastic properties; it binds to and stabilizes tubulin, thereby inhibiting microtubule disassembly which results in cell-cycle arrest at the G2/M phase and cell death. This agent also inhibits pro-angiogenic factors such as vascular endothelial growth factor (VEGF) and displays immunomodulatory and pro-inflammatory properties by inducing various mediators of the inflammatory response. Docetaxel has been studied for use as a radiation-sensitizing agent.

As used herein, the term “antimicrobial” refers to a substance, compound, or agent that kills or slows the growth of microbes, such as bacteria, fungi, viruses, or parasites. The term “antimicrobial agent” refers to a compound or agent with the ability to impede the growth of a microbe. Impeding growth further includes an agent which kills the microbe. For example, various antimicrobial agents act, inter alia, by interfering with (1) cell wall synthesis, (2) plasma membrane integrity, (3) nucleic acid synthesis, (4) ribosomal function, and (5) folate synthesis. In some embodiments, when the one or more hydrophobic agents is an antimicrobial agent, the antimicrobial agent is selected from the group consisting of doxycycline, cephalexin, gentamycin, kanamycin, rifamycins, novobiocin, and combinations thereof.

Doxycycline a synthetic, broad-spectrum tetracycline antibiotic exhibiting antimicrobial activity. Doxycycline binds to the 30S ribosomal subunit, possibly to the 50S ribosomal subunit as well, thereby blocking the binding of aminoacyl-tRNA to the mRNA-ribosome complex. This leads to an inhibition of protein synthesis. In addition, this agent has exhibited inhibition of collagenase activity.

Cephalexin is a beta-lactam, first-generation cephalosporin antibiotic with bactericidal activity. Cephalexin binds to and inactivates penicillin-binding proteins (PBP) located on the inner membrane of the bacterial cell wall. Inactivation of PBPs interferes with the cross-linking of peptidoglycan chains necessary for bacterial cell wall strength and rigidity. This results in the weakening of the bacterial cell wall and causes cell lysis. Compared to second and third generation cephalosporins, cephalexin is more active against gram-positive and less active against gram-negative organisms.

Gentamycin is a broad-spectrum aminoglycoside antibiotic produced by fermentation of Micromonospora purpurea or M. echinospora. Gentamycin is an antibiotic complex consisting of four major (C1, C1a, C2, and C2a) and several minor components. This agent irreversibly binds to the bacterial 30S ribosomal subunit. Specifically, this antibiotic is lodged between 16S rRNA and S12 protein within the 30S subunit. This leads to interference with translational initiation complex, misreading of mRNA, thereby hampering protein synthesis and resulting in bactericidal effect.

Kanamycin is an aminoglycoside antibiotic with antimicrobial property. Kanamycin irreversibly binds to the bacterial 30S ribosomal subunit, specifically in contact with 16S rRNA and S12 protein within the 30S subunit. This leads to interference with translational initiation complex and, misreading of mRNA, thereby hampering protein synthesis and resulting in bactericidal effect. This agent is usually used for treatment of E. coli, Proteus species (both indole-positive and indole-negative), E. aerogenes, K. pneumoniae, S. marcescens, and Acinetobacter species.

Rifamycin is a natural antibiotic produced by Streptomyces mediterranei, Rifamycin (Ansamycin Family) is a commonly used antimycobacterial drug that inhibits prokaryotic DNA-dependent RNA synthesis and protein synthesis; it blocks RNA-polymerase transcription initiation. Rifamycin has an activity spectrum against Gram-positive and Gram-negative bacteria, but is mainly used against Mycobacterium sp. (especially M. tuberculosis) in association with other agents to overcome resistance.

Novobicin is an aminocoumarin antibiotic, produced by the actinomycete Streptomyces nivens, with antibacterial property. Novobiocin, as well as other aminocoumarin antibiotics, inhibits bacterial DNA synthesis by targeting at the bacteria DNA gyrase and the related enzyme DNA topoisomerase IV. This antibiotic was used to treat infections by gram-positive bacteria.

Additional suitable hydrophobic agents include, without limitation, analgesics, anti-inflammatory agents, anthelmintics, antiarrhythmic agents, antibacterial agents, antiviral agents, anticogulantes, antidepressants, antidiabetics, antiepileptics, antifungal agents, anti-gout agents, antihypertensive agents, antimalarials, antimigraine agents, antimuscarinic agents, antineoplastic agents, erectile dysfunction improvement, immunosuppressants, antiprotozoal agents, antithyroid agents, anxiolytic agents, sedatives, hypnotics, neuroleptics, bloqueadores-beta, cardiac inotropic agents, corticosteroids, diuretics, antiparkinsonian agents, gastrointestinal agents, histamine receptor antagonists, keratolytics, lipid regulating agents, antianginal agents, Cox-2 inhibitors, leukotriene inhibitors, macrolides, muscle relaxants, agents nutrition signal, opioid analgesics, protease inhibitors, stimulants, muscle relaxants hormones, antiosteoporosis agents, antiobesity agents, cognitive enhancers, anti-urinary incontinence agents, antihipertrofia benign prostatic, essential fatty acids, non-essential fatty acids and mixtures thereof.

The one or more hydrophobic agents may include, without limitation, acitretin, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutamide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporine, danazol, dantrolene, dexchlorpheniramine, diclofenac, dicoumarol, digoxin, dehydroepiandrosterone, dihydroergotamine, dihydrotachysterol, dirithromycin, donezepyl, efavirenz, eprosartan, ergocalciferol, ergotamine, sources of essential fatty acids, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-triroxina, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nilsolidipina, nilutamide, nitrofurantoin, nizatidine, omeprazole, oprevelkin, oestradiol, oxaprozin, paclitaxel, paracalcitol, paroxetine, penta zocina, pioglitazone, pizofetin, pravastatin, prednisolone, probucol, progesterone, Pseudoephedrine, pyridostigmine, rabeprazole, raloxifene, rofecoxib, repaglinide, rifabutin, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibrano, tizanidine, topiramate, topotecan, toremifene, tramadol, tretinoin, troglitazone, trovafloxacin, ubidecarenone, valsartan, venlafaxine, verteporfin, vigabatrin, zafirlukast, zileuton, zolmitriptan, zolpidem, zopiclone, pharmaceutically acceptable salts, isomers and derivatives thereof and mixtures thereof.

In some embodiments, one or more hydrophobic agents is a treatment for Alzheimer's Disease such as Aricept and Excelon, a treatment for Parkinson's Disease such as L-DOPA/carbidopa, entacapone, ropinirole, pramipexole, bromocriptine, pergolide, trihexyphenidyl or amantadine; an agent for treating Multiple Sclerosis (MS) such as beta interferon (e.g., Abonex® and Rebif®), Copaxona® or mitoxantrone; treatment for asthma, such as a steroid, albuterol or Singulair®; an agent for treating schizophrenia such as zyprexa, risperdal, seroquel or haloperidol; an antiinflammatory agent such as corticosteroids, TNF blockers, IL-1 RA, azathioprine, cyclophosphamide or sulfasalazine; immunomodulatory and immunosuppressive agent one as cyclosporin, tacrolimus, rapamycin, mycophenolate mofetil, interferons, corticosteroids, cyclophosphamide, azathioprine or sulfasalazine; a neurotrophic factor such as acetylcholinesterase inhibitors, MAO inhibitors, interferons, anticonvulsants, ion channel blockers, riluzole or antiparkinsonian agents; an agent for treating cardiovascular disease such as beta-blockers, ACE inhibitors, diuretics, nitrates, calcium channel blockers, or statins; an agent for treating liver disease such as corticosteroids, cholestyramine, interferons, or antiviral agents; an agent for treating blood disorders such as corticosteroids, anti-leukemia agents, or growth factors; and an agent for treating immunodeficiency disorders such as gamma globulin.

In some embodiments, the one or more hydrophobic agents is an imaging agent selected from the group consisting of Nile red, pyrene, anthracene, and derivatives and combinations thereof.

Nile red is phenoxazone dye that fluoresces intensely, and in varying color, in organic solvents and hydrophobic lipids (Fowler et al., “Application of Nile red, a Fluorescent Hydrophobic Probe, for the Detection of Neutral Lipid Deposits in Tissue Sections: Comparison with Oil Red O,” J. Histochem. Cytochem. 33(8):833-836 (1985), which is hereby incorporated by reference in its entirety).

Pyrene is a polycyclic aromatic hydrocarbon consisting of four fused benzene rings, resulting in a flat aromatic system. Pyrene and its derivatives are used commercially to make dyes and dye precursors including, e.g., pyranine and naphthalene-1,4,5,8-tetracarboxylic acid.

Anthracene, also called paranaphthalene or green oil, a solid polycyclic aromatic hydrocarbon (PAH) consisting of three benzene rings derived from coal-tar, is the simplest tricyclic aromatic hydrocarbon and is primarily used as an intermediate in the production of dyes, smoke screens, scintillation counter crystals, and in organic semiconductor research.

The hydrophilic exterior of the encapsulated product may be covalently modified to comprise one or more targeting agents. As described herein, the “one or more targeting agents” serve to enhance the pharmacokinetic or bio-distribution properties of the compound to which they are linked, and improve cell-specific or tissue-specific distribution and cell-specific uptake of the conjugated composition. The one or more targeting agents aid in directing the delivery of the encapsulated product to which it is linked to the desired target site. In some embodiments, the one or more targeting agents binds to a cell or cell receptor, and initiate endocytosis to facilitate entry of the therapeutic compound into the cell. Targeting agents include, without limitation, compounds with affinity to cell receptors or cell surface molecules or antibodies.

Suitable targeting agents include, without limitation, hydrophilic polymers selected from the group consisting of polyethylene glycol (PEG), polysialic acid (PSA), polylactic (i.e., polylactide), polyglycolic acid (i.e., polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses (e.g., hydroxymethylcellulose, hydroxyethylcellulose), hyaluronic acid (HA), and derivatives thereof (see, e.g., Pasut, G., “Polymers for Protein Conjugation,” Polymers 6:160-178 (2014), which is hereby incorporated by reference in its entirety).

In some embodiments, the one or more targeting agents is a polymer selected from the group consisting of hyaluronic acid (HA), polysialic acid (PSA), polyethylene glycol (PEG), and combinations thereof.

Hyaluronic acid is a glucosaminoglycan consisting of D-glucuronic acid and N-acetyl-D-glucosamine disaccharide units that is a component of connective tissue, skin, vitreous humour, umbilical cord, synovial fluid and the capsule of certain microorganisms contributing to adhesion, elasticity, and viscosity of extracellular substances.

Polysialic acid is a highly negative-charged carbohydrate composed of a linear polymer of alpha 2,8-linked sialic acid residue with potential immunotherapeutic activity. Polysialic acid (PSA) is mainly attached to the neural cell adhesion molecule (NCAM), a membrane-bound glycoprotein overexpressed in certain types of cancers. In embryonic tissue, PSA-NCAM is abundantly expressed and PSA plays an important role in formation and remodeling of the neural system through modulation of the adhesive properties of NCAM, thereby reducing cell-cell interactions and promoting cellular mobility. In adult tissue however, the expression of PSA-NCAM is associated with a variety of malignant tumors, signifying its potential role in tumor metastasis.

Polyethylene glycol is a polymer made by joining molecules of ethylene oxide and water together in a repeating pattern. Polyethylene glycol can be a liquid or a waxy solid.

In some embodiments, the one or more targeting agents is an antibody or binding fragment thereof. As used herein, the term “antibody” refers to any specific binding substance(s) having a binding domain with a required specificity including, but not limited to, antibody fragments, derivatives, functional equivalents, and homologues of antibodies, including any polypeptide comprising an immunoglobulin binding domain, whether natural or synthetic, monoclonal or polyclonal. The antibody may be a human antibody selected from the group consisting of IgG, IgA, IgM, and IgE. In some embodiments, the antibody is an IgG antibody. Suitable antibody binding fragments include, without limitation, Fab fragments, F(ab)₂ fragments, Fab′ fragments, F(ab′)₂ fragments, Fd fragments, Fd′ fragments, or Fv fragments.

In some embodiments, the one or more targeting agents is a peptide targeting agent. Suitable peptide targeting agents are well known in the art and include, without limitation, Octreotide, RC160, Bombesin, PSAP-peptide, NT21MP, Nef-M1, Peptide R, Pentixafor, pHLIP, L-zipper peptide, ELP, α-MSH mimics, GZP, cRGD, EETI 2.5 F (knottin), NGR, SP2012, AARP, CK, LyP-1, AGR, REA, LSD, iRGD, iPhage/pen, M2pep, CooP, CLT-1, Pep-1 L, Angiopep-2, Angiopep-7, FHK, tLyP-1, and Cilengitide (LeJoncour et al., “Seek & Destroy, Use of Targeting Peptides for Cancer Detection and Drug Delivery,” Bioorganic & Medicinal Chemistry 26:2797-2806 (2018), which is hereby incorporated by reference in its entirety).

In some embodiments, the one or more targeting agents is an aptamer. As used herein, the term “aptamer” or “aptamers” refers to single-stranded DNA or RNA oligonucleotides that bind their targets with high affinity and selectivity (U.S. Pat. No. 9,688,991 to Levy et al. and Lee et al., “Conjugation of Prostate Cancer-Specific Aptamers to Polyethylene Glycol-Grafted Polyethylenimine for Enhanced Gene Delivery to Prostate Cancer Cells,” Journal of Industrial and Engineering Chemistry 73:182-191 (2019), which are hereby incorporated by reference in their entirety).

Additional suitable targeting agents may be selected from the group consisting of receptor-binding ligands, such as hormones or other molecules that bind specifically to a receptor; cytokines, which are polypeptides that affect cell function and modulate interactions between cells associated with immune, inflammatory or hematopoietic responses; molecules that bind to enzymes, such as enzyme inhibitors; nucleic acid ligands, and one or more members of a specific binding interaction such as biotin or iminobiotin and avidin or streptavidin.

The one or more targeting agents may be specific to a cancer-specific antigen. Thus, in some embodiments, the antibody or derivative thereof is specific to a breast cancer antigen, a lung cancer antigen, a colon cancer antigen, an ovarian cancer antigen, a prostate cancer antigen, or a kidney cancer antigen (see, e.g., U.S. Pat. No. 7,560,095 to Sun et al.; U.S. Pat. No. 7,485,300 to Young et al.; and U.S. Pat. No. 5,171,665 to Hellstrom et al., which are hereby incorporated by reference in their entirety).

As demonstrated herein, both aromatic amino acids, such as L-histidine, and non-aromatic amino acids, such as L-glutamine, L-isoleucine, L-asparagine, L-valine, L-threonine, and L-methionine, show fluorescence emission upon crystallization in the solid state. Thus, in some embodiments, the crystal is fluorescent. Such fluorescent encapsulated products can be used in bioimaging, chemosensing, optoelectronics, and stimuli-responsive systems (Mei et al., “Aggregation-Induced Emission: Together We Shine, United We Soar!,” Chem. Rev 115(21):11718-11940 (2015); Ravanfar et al., “Controlling the Release From Enzyme-Responsive Microcapsules With a Smart Natural Shell,” ACS Applied Materials & Interfaces 10(6):6046-6053 (2018); Ravanfar et al., “Thermoresponsive, Water-Dispersible Microcapsules With a Lipid-Polysaccharide Shell To Protect Heat-Sensitive Colorants,” Food Hydrocolloids 81:419-428 (2018); and Ravanfar et al., “Preservation of Anthocyanins in Solid Lipid Nanoparticles: Optimization of a Microemulsion Dilution Method Using the Placket-Burman and Box-Behnken Designs,” Food Chemistry 199:573-580 (2016), which are hereby incorporated by reference in their entirety).

Another aspect of the present application relates to a pharmaceutical or cosmetic composition comprising a pharmaceutically or cosmetically acceptable carrier and the encapsulated product as described herein.

The term “pharmaceutically or cosmetically acceptable carrier” refers to a carrier that does not cause an allergic reaction or other untoward effect in patients to whom it is administered and are compatible with the other ingredients in the formulation. Pharmaceutically or cosmetically acceptable carriers include, for example, pharmaceutical or cosmetic diluents, excipients or carriers suitably selected with respect to the intended form of administration, and consistent with conventional pharmaceutical or cosmetic practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically or cosmetically acceptable carriers may further comprise minor amounts of auxiliary substances such as wetting or emulsifying agents, preservatives or buffers, which enhance the shelf life or effectiveness of the encapsulated product.

In certain embodiments, the pharmaceutical or cosmetically acceptable carrier is an aqueous medium that is well tolerated for administration to an individual, typically a sterile isotonic aqueous buffer. Exemplary aqueous media include, without limitation, normal saline (about 0.9% NaCl), phosphate buffered saline (PBS), sterile water/distilled autoclaved water (DAW), as well as cell growth medium (e.g., MEM, with or without serum), aqueous solutions of dimethyl sulfoxide (DMSO), polyethylene glycol (PEG), and/or dextran (less than 6% per by weight).

To improve patient tolerance to administration, the pharmaceutical or cosmetic composition preferably has a pH of about 6 to about 8, preferably about 6.5 to about 7.4. Typically, sodium hydroxide and hydrochloric acid are added as necessary to adjust the pH.

The pharmaceutical or cosmetic composition suitably includes a weak acid or salt as a buffering agent to maintain pH. Citric acid has the ability to chelate divalent cations and can thus also prevent oxidation, thereby serving two functions as both a buffering agent and an antioxidant stabilizing agent. Citric acid is typically used in the form of a sodium salt, typically 10-500 mM. Other weak acids or their salts can also be used.

The pharmaceutical or cosmetic composition may also include solubilizing agents, preservatives, stabilizers, emulsifiers, and the like. A local anesthetic (e.g., lidocaine) may also be included in the compositions, particularly for injectable forms, to ease pain at the site of the injection.

The pharmaceutical composition described herein may be suitable for administration orally, topically, transdermally, parenterally, intradermally, intrapulmonary, intramuscularly, intraperitoneally, intravenously, subcutaneously, or by intranasal instillation, by intracavitary or intravesical instillation, intraocularly, intraarterialy, intralesionally, or by application to mucous membranes.

The cosmetic composition described herein may be suitable for administration topically.

Suitable compositions for topical administration include, without limitation, a cream, an ointment, a gel, a paste, a powder, a spray, a suspension, a dispersion, a salve, and a lotion.

As demonstrated herein, entrapment of hydrophobic small molecules inside the hydrophobic domains of amino acid crystals provides a platform for protecting hydrophobic agents (e.g., vitamins, carotenoids, antioxidants, drugs, imaging agents, and combinations thereof). In some embodiments, the one or more hydrophobic agents is present at about 0.01-99% w/w (e.g., 0.01-99%, 0.01-90%, 0.01-85%, 0.01-80%, 0.01-75%, 0.01-70%, 0.01-65%, 0.01-60%, 0.01-55%, 0.01-50%, 0.01-45%, 0.01-40%, 0.01-35%, 0.01-30%, 0.01-25%, 0.01-20%, 0.01-15%, 0.01-10%, 0.01-5%, 0.01-0.1%, 0.1-99%, 0.1-90%, 0.1-85%, 0.1-80%, 0.1-75%, 0.1-70%, 0.1-65%, 0.1-60%, 0.1-55%, 0.1-50%, 0.1-45%, 0.1-40%, 0.1-35%, 0.1-30%, 0.1-25%, 0.1-20%, 0.1-15%, 0.1-10%, 0.1-5%, or 0.1-1%). In some embodiments, the one or more hydrophobic agents is present at a concentration having a lower limit selected from 0.01%, 0.05%, 0.10%, 0.15%, 0.20%, 0.25%, 0.30%, 0.35%, 0.50%, 0.55%, 0.60%, 0.65%, 0.70%, 0.75%, 0.80%, 0.85%, 0.90%, 0.95%, 1%, 2%, 3%, 4%, 5%, 6%, 7%, 8%, 9%, 10%, 15%, 20%, 25%, 30%, 40%, 50%, 55%, 60%, 65%, 70%, 75%, 80%, 85%, 90%, 95%, and an upper limit selected from 0.05%, 0.10%, 0.15%, 0.20%, 0.25%, 0.30%, 0.35%, 0.50%, 0.55%, 0.60%, 0.65%, 0.70%, 0.75%, 0.80%, 0.85%, 0.90%, 0.95%, 1%, 2%, 3%, 4%, 5%, 6%, 7%, 8%, 9%, 10%, 15%, 20%, 25%, 30%, 40%, 50%, 55%, 60%, 65%, 70%, 75%, 80%, 85%, 90%, 95%, 96%, 97%, 98%, 99%, or more, and any combination thereof. For example, the hydrophobic agent may be present at a concentration of about 0.1-65% w/w.

A further aspect of the present application relates to a method of therapeutically treating a subject with one or more hydrophobic agents. This method involves selecting a subject in need of therapeutic treatment and administering the encapsulated product or pharmaceutical or cosmetic composition described herein to the selected subject.

In carrying out the methods of the present application, “treating” or “treatment” includes inhibiting, ameliorating, or delaying onset of a particular condition or state. Treating and treatment also encompasses any improvement in one or more symptoms of the condition or disorder. Treating and treatment encompasses any modification to the condition or course of disease progression as compared to the condition or disease in the absence of therapeutic intervention.

In some embodiments, the subject is in need of treatment for cancer.

The cancer may be selected from the group consisting of adrenal cortical cancer, anal cancer, aplastic anemia, bile duct cancer, bladder cancer, bone cancer, bone metastasis, central nervous system (CNS) cancers, peripheral nervous system (PNS) cancers, Castleman's disease, cervical cancer, colon and rectum cancer, endometrial cancer, esophagus cancer, Ewing's family of tumors (e.g., Ewing's sarcoma), eye cancer, gallbladder cancer, gastrointestinal carcinoid tumors, gastrointestinal stromal tumors, gestational trophoblastic disease, hairy cell leukemia, Hodgkin's disease, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, thyroid cancer, uterine cancer (e.g. uterine sarcoma), vaginal cancer, vulvar cancer, and Waldenstrom's macroglobulinemia.

The selected subject may be in need of treatment for cancer. In accordance with these embodiments, the encapsulated product comprises one or more anticancer agents. Suitable anticancer agents are described in detail above. For example, in some embodiments, when the selected subject is in need of treatment for breast cancer, the one or more hydrophobic agents may be selected from the group consisting of doxorubicin HCl, paclitaxel, 5-fluorouracil, camptothecin, cisplatin, metronidazole, melphalan, docetaxel, and derivatives and combinations thereof.

The selected subject may be in need of treatment for a vitamin deficiency. As described herein, vitamin A deficiency may result from inadequate intake, fat malabsorption, or liver disorders. Vitamin A deficiency impairs immunity and hematopoiesis and causes rashes and typical ocular effects (e.g., xerophthalmia, night blindness) (see, e.g., Porter, Robert S, and Justin L. Kaplan. The Merck Manual of Diagnosis and Therapy, 2018, which is hereby incorporated by reference in its entirety). Vitamin D deficiency impairs bone mineralization, causing rickets in children and osteomalacia in adults and possibly contributing to osteoporosis (see, e.g., Porter, Robert S, and Justin L. Kaplan. The Merck Manual of Diagnosis and Therapy; 2018, which is hereby incorporated by reference in its entirety). Symptoms of vitamin E deficiency include hemolytic anemia and neurologic deficits (see, e.g., Porter, Robert S, and Justin L. Kaplan. The Merck Manual of Diagnosis and Therapy, 2018, which is hereby incorporated by reference in its entirety). Vitamin K deficiency impairs clotting (see, e.g., Porter, Robert S, and Justin L. Kaplan. The Merck Manual of Diagnosis and Therapy. 2018, which is hereby incorporated by reference in its entirety).

The vitamin deficiency may be selected from the group consisting of vitamin A deficiency, vitamin D deficiency, vitamin E deficiency, vitamin K deficiency, and combinations thereof. In accordance with these embodiments, the encapsulated product may comprise one or more vitamins selected from the group consisting of vitamin A, vitamin D, vitamin E, vitamin K, and combinations thereof.

The selected subject may be in need of treatment for a sleep disorder. In accordance with these embodiments, the one or more hydrophobic agents comprises melatonin.

The selected subject may be in need of an antioxidant. In accordance with these embodiments, the one or more hydrophobic agents comprises melatonin, vitamin A, and vitamin E.

The selected subject may be in need of treatment for a disease selected from the group consisting of a dermatological disorder, dermatological disease, or dermatological imperfection.

Exemplary skin diseases include, without limitation, scabies, eczema, melisma, pityriasis versicolor, and acne.

Exemplary dermatological disorders include, without limitation, rosacea, acne, pityriasis rosea, inflammatory skin reactions such as urticaria (swelling with raised edges), general swelling, and erythema. Suitable dermatological imperfections include, without limitation, macules, papules, plaques, nodules, vesicles, bullae, pustules, urticarial, scales, scabs, erosions, ulcers, petachiae, purpura, atrophy, scars, hyperpigmentation, and telangiectases.

The selected subject may be in need of treatment for an infectious disease. As used herein, the term “infectious disease” refers to a clinically evident disease resulting from the presence of pathogenic microbial agents, including pathogenic viruses, pathogenic bacteria, fungi, protozoa, multicellular parasites, and aberrant proteins known as prions. Infectious pathologies are usually qualified as contagious diseases (also called communicable diseases) due to their potentiality of transmission from one person or species to another. Transmission of an infectious disease may occur through one or more of diverse pathways including physical contact with infected individuals. These infecting agents may also be transmitted through liquids, food, body fluids, contaminated objects, airborne inhalation, or through vector-borne spread.

In some embodiments, when the subject is in need of treatment for an infectious disease, the encapsulated product comprises one or more antimicrobial agents. Suitable antimicrobial agents are described in detail above and include, e.g., doxycycline, cephalexin, gentamycin, kanamycin, rifamycin, novobiocin, and derivatives and combinations thereof.

Suitable subjects in accordance with the methods described herein include, without limitation, mammals. In some embodiments, the subject is selected from the group consisting of primates (e.g., humans, monkeys), equines (e.g., horses), bovines (e.g., cattle), porcines (e.g., pigs), ovines (e.g., sheep), caprines (e.g., goats), camelids (e.g., llamas, alpacas, camels), rodents (e.g., mice, rats, guinea pigs, hamsters), canines (e.g., dogs), felines (e.g., cats), leporids (e.g., rabbits). In some embodiments, the selected subject is an agricultural animal, a domestic animal, or a laboratory animal. In some embodiments, the subject is a human subject. Suitable human subjects include, without limitation, infants, children, adults, and elderly subjects.

Yet another aspect of the present application relates to a method of in vitro imaging. This method involves contacting the in vitro cell culture system with the encapsulated product or pharmaceutical or cosmetic composition described herein and imaging the contacted cell culture system.

The in vitro culture system may comprise mammalian cells selected from the group consisting of primate cells (e.g., human cells, monkey cells), equine cells (e.g., horse cells), bovine cells (e.g., cattle cells), porcine cells (e.g., pig cells), ovine cells (e.g., sheep cells), caprine cells (e.g., goat cells), camelid cells (e.g., llama cells, alpaca cells, camel cells), rodent cells (e.g., mice cells, rat cells, guinea pig cells, hamster cells), canine cells (e.g., dog cells), feline cells (e.g., cat cells), and leporid cells (e.g., rabbit cells). Thus, the cells may be human cells.

In some embodiments, the in vitro cell culture system comprises a population of primary cells (e.g., a tissue sample). As used herein, the term “primary cells” refers to cells which have been isolated directly from human or animal tissue. Once isolated, they are placed in an artificial environment in plastic or glass containers supported with specialized medium containing essential nutrients and growth factors to support proliferation. Primary cells may be adherent or suspension cells. Adherent cells require attachment for growth and are said to be anchorage-dependent cells. The adherent cells are usually derived from tissues of organs. Suspension cells do not require attachment for growth and are said to be anchorage-independent cells.

In some embodiments, the in vitro cell culture system comprises a population of cell line cells. As used herein, the term “cell line cells” refers to cells that have been continuously passaged over a long period of time and have acquired homogenous genotypic and phenotypic characteristics. Cell lines can be finite or continuous. An immortalized or continuous cell line has acquired the ability to proliferate indefinitely, either through genetic mutations or artificial modifications. A finite cell line has been sub-cultured for 20-80 passages after which the cells have senesced. Suitable cell line cells include, without limitation, HeLa, HEK293, HEK293T, MCF-7, MDA-MB-157, MDA-MB-231, MFM-223, CHO, 3T3, A549, and Vero cell lines. In some embodiments, the cell line cells are tumor cell line cells.

Imaging the contacted cell culture system may be carried out using ultraviolet-visible (UV-VIS) spectroscopy and/or fluorescence spectroscopy (e.g., single molecule fluorescence microscopy, fluorescence correlation spectroscopy, confocal microscopy, multiphoton microscopy, total internal reflection microscopy, and combinations thereof) (see, e.g., Combs, C., “Fluorescence Microscpy: A Concise Guide to Current Imaging Methods,” Curr. Protocol. Neurosci. 2:Unit 2.1 (2013), which is hereby incorporated by reference in its entirety).

As described herein, confocal microscopy achieves very high resolution by using the same objective lens to focus both a parallel beam of incident light and the resulting emitted light at the same small spot on or near the surface of target tissue.

As described herein, the encapsulated product may be modified to comprise one or more targeting agents, e.g., hyaluronic acid (HA). In tumor tissues, HA is contributed by both tumor stroma and tumor cells and induces intracellular. Thus, HA may be used to target the encapsulated product to tumor cells (Lokeshwar et al., “Targeting Hyaluronic Acid Family for Cancer Chemoprevention and Therapy,” Adv. Cancer Res. 123:35-65 (2014), which is hereby incorporated by reference in its entirety). Covalently cross-linking HA to the surface of the encapsulated product may be carried out such that hyaluronidase (HAase) in a target cell hydrolyzes the HA to allow the crystals of the encapsulated target to dissolve and release the one or more entrapped hydrophobic agents. Accordingly, the methods of in vitro imaging described herein may be utilized to detect the delivery of the one or more entrapped hydrophobic agents to a target cell.

In the context of the methods described herein, the administering, contacting, and/or imaging steps may be repeated. For example, the administering or contacting may be carried out at least 2, 3, 4, 5, 6, 7, 8, 9, 10, or more times.

In some embodiments, the administering, contacting, and/or imaging is carried out daily, weekly, or monthly. For example, the administering, contacting, and/or imaging steps can be carried out daily for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days. In some embodiments, the administering, contacting, and/or imaging can be carried out weekly for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more weeks. In other embodiments, the administering, contacting, and/or imaging can be carried out monthly for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more months.

In some embodiments, the method of in vitro imaging further involves allowing the encapsulated product or pharmaceutical or cosmetic composition described herein to bind a target cell prior to or during the imaging step. Conditions under which the encapsulated product may bind to its target cell are empirically determined by one of ordinary skill in the art by varying certain parameters, e.g., salt concentrations, pH, temperature, concentration of the target, concentration of the biological agent. A skilled scientist would appreciate that these parameters affect the binding of the encapsulated product to the target. Typically, but not always, suitable conditions for allowing the encapsulated product to bind to the target cell are physiological conditions, such that in the methods of therapeutically treating a subject described herein, suitable conditions may be providing a sufficient period of time for the encapsulated product to bind to the target cell.

In some embodiments, the imaging is carried out to detect the presence or absence of the encapsulated product or pharmaceutical or cosmetic composition. Thus, the imaging may be carried out to monitor the delivery of the encapsulated product.

Another aspect of the present application relates to a method of preparing an encapsulated product comprising entrapped hydrophobic agents. This method involves mixing one or more hydrophobic agents with one or more amino acids to produce a mixture and forming crystals of the one or more amino acids entrapping the one or more hydrophobic agents, where the crystals have a hydrophilic exterior.

In some embodiments, the mixing is carried out in an aqueous solution of one or more amino acids.

The encapsulated products of the present application can be synthesized using standard crystallization techniques, which are well known to those of ordinary skill in the art (see, e.g., McPherson et al., “Introduction to Protein Crystallization,” Acta. Crystallogr. F. Struct. Biol. Commun. 70(Pt 1):2-20 (2014), which is hereby incorporated by reference in its entirety). These include, e.g., slow cooling, ultrasonic agitation, sublimation, vapor diffusion, dialysis crystallization, antisolvent crystallization, and solvent evaporation (U.S. Pat. No. 5,118,815 to Shiroshita et al. and U.S. Pat. No. 7,378,545 to Bechtel et al., each of which are hereby incorporated by reference in their entirety). In general, crystallization involves nucleation, crystal growth and cessation of growth (see, e.g., Krauss et al., “An Overview of Biological Macromolecule Crystallization,” Int. J. Mol. Sci. 14(6): 11643-11691 (2013), which is hereby incorporated by reference in its entirety). During nucleation an adequate amount of molecules associate in three dimensions to form a thermodynamically stable aggregate, the so called critical nucleus, which provides surfaces suitable for crystal growth. The growth stage, which immediately follows the nucleation, is governed by the diffusion of particles to the surface of the critical nuclei and their ordered assembling onto the growing crystal. Protein crystal formation requires interactions that are specific, highly directional and organized in a manner that is appropriate for three-dimensional crystal lattice formation. Crystal growth ends when the solution is sufficiently depleted of protein molecules, deformation-induced strain destabilizes the lattice, or the growing crystal faces become poisoned by impurities. The crystallizability of a protein is strictly affected by the chemical and conformational purity and the oligomeric homogeneity of the sample.

As used herein, slow cooling involves dissolving the one or more amino acids and the one or more hydrophobic agents in a minimum amount of a hot solvent and allowing the resulting solution to cool slowly to room temperature.

As used herein, ultrasonic agitation involves subjecting a solution of the one or more amino acids and the one or more hydrophobic agents to ultrasonic agitation at a temperature and for a period of time sufficient to produce a crystal of the one or more amino acids entrapping the one or more hydrophobic agents.

As use herein, sublimation involves heating a solution of one or more amino acids and the one or more hydrophobic agents under reduced pressure until it vaporizes and allowing it to undergo deposition onto a cool surface to form a crystal.

As used herein, vapor diffusion is a crystallization method that utilizes evaporation and diffusion of water (and other volatile species between a small droplet (0.5-10 μl), containing protein, buffer and precipitant, and a reservoir (well), containing a solution with similar buffer and precipitant, but at higher concentrations with respect to the droplet (Krauss et al., “An Overview of Biological Macromolecule Crystallization,” Int. J. Mol. Sci. 14(6): 11643-11691 (2013), which is hereby incorporated by reference in its entirety). The wells are sealed by creating an interface of vacuum grease between the rim of each well and the cover slip, or by using, in specific cases, a sealing tape. The droplet is equilibrated over the well solution as either a hanging, a sitting or a sandwich drop to allow a slow increase of both the protein and precipitant concentration that could cause supersaturation and crystal growth. In the hanging method, the drop is placed on the underside of a siliconized glass cover slide, while in the sitting method, the drop is placed on a plastic or glass support above the surface of the reservoir. Finally in the sandwich drop, the protein mixed with the precipitant is placed between two cover slips, one of which closes the well. The difference between the concentration of the precipitant in the drop and in the well solution causes the evaporation of water from the drop until the concentration of the precipitant equals that of the well solution. Since the volume of the well solution is much larger (500-1000 μL) than the volume of the drop (few microliters), its dilution by the water vapor leaving the droplet is negligible.

As used herein, dialysis crystallization utilizes diffusion and equilibration of precipitant molecules through a semi-permeable membrane as a means of slowly approaching the concentration at which the macromolecule crystallizes. Provided that the precipitant is a small molecule like a salt or an alcohol, it can easily penetrate the dialysis membrane, and the protein is slowly brought into equilibrium with the precipitant solution.

As used herein, antisolvent crystallization reduces the solubility of a solute in the solution and to induce rapid crystallization.

In some embodiments, the mixing is carried out in an aqueous solution. Aqueous solutions may include, without limitation, dimethyl sulfoxide (DMSO), polyethylene glycol (PEG), and/or dextran.

In some embodiments, the mixing and incubating steps are carried out at a temperature of 0° C.-60° C. (e.g., 0-60° C., 5-60° C., 10-60° C., 15-60° C., 20-60° C., 25-60° C., 30-60° C., 35-60° C., 40-60° C., 45-60° C., 50-60° C., 55-60° C., 0-55° C., 0-50° C., 0-45° C., 0-40° C., 0-35° C., 0-30° C., 0-25° C., 0-20° C., 0-15° C., 0-10° C., or 0-5° C.). In some embodiments, the mixing and incubating steps are carried out at temperature having a lower limit selected from 0° C., 5° C., 10° C., 15° C., 20° C., 25° C., 30° C., 35° C., 40° C., 45° C., 50° C., 55° C., and an upper limit selected from 5° C., 10° C., 15° C., 20° C., 25° C., 30° C., 35° C., 40° C., 45° C., 50° C., 55° C., and 60° C., and any combination thereof.

The one or more amino acids are aromatic, non-aromatic, or combinations thereof. Suitable aromatic amino acids, non-aromatic amino acids, and combinations of aromatic and non-aromatic amino acids are described in detail above. For example, the aromatic amino acids may be selected from the group consisting of histidine, phenylalanine, tyrosine, and tryptophan. The non-aromatic amino acids are selected from the group consisting of glutamine, isoleucine, asparagine, valine, threonine, and methionine.

In some embodiments, the one or more amino acids are L-amino acids, D-amino acids, or combinations thereof. Suitable L-amino acids, D-amino acids, and combinations of L-amino acids and D-amino acids are described in detail above. In some embodiments, the one or more amino acids is L-histidine.

In some embodiments, the one or more amino acids are monomers, dimers, trimers, or combinations thereof. Suitable monomers, dimers, and trimers are described in detail above.

The one or more hydrophobic agents may be selected from the group consisting of vitamins, carotenoids, antioxidants, drugs, imaging agents, and combinations thereof Suitable vitamins, carotenoids, antioxidants, drugs, and imaging agents are described in detail above.

The use of the antisolvent in crystallization reduces the solubility of a solute in the solution and to induce rapid crystallization. The physical and chemical properties of the anti-solvent can alter the rate of mixing with the solutions and thereby affect the rate of nucleation and crystal growth of the crystallizing compounds.

In some embodiments, the mixture further comprises an antisolvent. The antisolvent may be selected from the group consisting of ethanol, methanol, Tetrahydrofuran, acetone, and combinations thereof.

In some embodiments, the crystal is formed by cooling the mixture of the one or more hydrophobic agents with one or more amino acids.

The method of forming the encapsulated product may further involve washing the crystals to remove unentrapped hydrophobic agents and modifying the washed crystals' surfaces to include a targeting agent.

Suitable targeting agents are described in detail above. For example, the targeting agent may be a polymer selected from the group consisting of hyaluronic acid (HA), polysialic acid (PSA), polyethylene glycol (PEG), and combinations thereof.

As demonstrated herein, the entrapment efficiency (i.e., the concentration of the entrapped one or more hydrophobic agents within the encapsulated product as compared to the concentration of the non-entrapped one or more hydrophobic agents) can be calculated using the formula in equation 1:

Entrapment efficiency %=M₀−MₛM₀*100 (1),
where M₀ is the primary concentration of small molecules used in the formulation, and Mₛ is the concentration of non-entrapped small molecules in the supernatant. In some embodiments, the entrapment efficiency of the one or more hydrophobic agents is in the range of about 0.01-99% (e.g., 0.01-99%, 0.01-90%, 0.01-85%, 0.01-80%, 0.01-75%, 0.01-70%, 0.01-65%, 0.01-60%, 0.01-55%, 0.01-50%, 0.01-45%, 0.01-40%, 0.01-35%, 0.01-30%, 0.01-25%, 0.01-20%, 0.01-15%, 0.01-10%, 0.01-5%, 0.01-0.1%, 0.1-99%, 0.1-90%, 0.1-85%, 0.1-80%, 0.1-75%, 0.1-70%, 0.1-65%, 0.1-60%, 0.1-55%, 0.1-50%, 0.1-45%, 0.1-40%, 0.1-35%, 0.1-30%, 0.1-25%, 0.1-20%, 0.1-15%, 0.1-10%, 0.1-5%, or 0.1-1%). In some embodiments, the entrapment efficiency has a lower limit selected from 0.01%, 0.05%, 0.10%, 0.15%, 0.20%, 0.25%, 0.30%, 0.35%, 0.50%, 0.55%, 0.60%, 0.65%, 0.70%, 0.75%, 0.80%, 0.85%, 0.90%, 0.95%, 1%, 2%, 3%, 4%, 5%, 6%, 7%, 8%, 9%, 10%, 15%, 20%, 25%, 30%, 40%, 50%, 55%, 60%, 65%, 70%, 75%, 80%, 85%, 90%, 95%, and an upper limit selected from 0.05%, 0.10%, 0.15%, 0.20%, 0.25%, 0.30%, 0.35% 0.50%, 0.55%, 0.60%, 0.65%, 0.70%, 0.75%, 0.80%, 0.85%, 0.90%, 0.95%, 1%, 2%, 3%, 4%, 5%, 6%, 7%, 8%, 9%, 10%, 15%, 20%, 25%, 30%, 40%, 50%, 55%, 60%, 65%, 70%, 75%, 80%, 85%, 90%, 95%, 96%, 97%, 98%, 99%, or more, and any combination thereof. For example, the entrapment efficiency may be in the range of about 0.1-65%.

The present application may be further illustrated by reference to the following examples.

### EXAMPLES

The examples below are intended to exemplify the practice of embodiments of the disclosure but are by no means intended to limit the scope thereof.

### Materials and Methods for Examples 1-7

Preparation and Characterization of the L-his Crystals with Entrapped Small Molecules

A 30 mg/mL solution of L-His (>99%, Sigma-Aldrich) was prepared by dissolving L-His powder in milli-Q water using a vortex mixer at ambient temperature in a Corning® 15 mL centrifuge tube with a closed cap. Then 500 μL of the aqueous solution of L-His and 500 μL of 200 proof ethanol (KOPTEC, PA, US) was added to 200 μL of the small molecule solution (2 mg/mL). The small molecules used in this study were Nile red (>98%, Sigma-Aldrich), pyrene (>98%, Sigma-Aldrich), β-carotene (>97%, Sigma-Aldrich), and doxorubicin HCl (DOX, >98%, Fluka, Mexico City, Mexico). The solution was vortexed for 15 seconds and kept static at ambient temperature. After 3 hours, crystals were collected and washed with ethanol to remove the free small molecules from the surface of the crystals and the supernatant was collected to measure the concentration of non-entrapped small molecules using HPLC. An Agilent 1200 LC System with a Binary SL Pump & Diode Array Detector, Shodex RI-501 Refractive Index Detector (single channel), and an Agilent 1100 Column Compartment (G1316) was utilized to carry out the analysis. Each individual sample of small molecules was quantified based on an optimized method reported in the literature for β-carotene (Etzbach et al., “Characterization of Carotenoid Profiles in Goldenberry (Physalis peruviana L.) Fruits at Various Ripening Stages and in Different Plant Tissues by HPLC-DAD-APCI-MSⁿ,” Food Chem. 245:508-517 (2018), which is hereby incorporated by reference in its entirety), Nile red (Wu et al., “Drug Delivery to the Skin from Sub-Micron Polymeric Particle Formulations: Influence of Particle Size and Polymer Hydrophobicity,” Pharm Res. 26(8):1995-2001 (2009), which is hereby incorporated by reference in its entirety), pyrene (Jia et al., “Effect of Root Exudates on the Mobility of Pyrene in Mangrove Sediment-Water System,” Catena 162:396-401 (2017), which is hereby incorporated by reference in its entirety), and DOX (Chi et al., “Redox-Sensitive and Hyaluronic Acid Functionalized Liposomes for Cytoplasmic Drug Delivery to Osteosarcoma in Animal Models,” J. Control Release 261:113-125 (2017), which is hereby incorporated by reference in its entirety). The entrapment efficiency of the crystals was calculated by subtracting the concentration of the non-entrapped small molecules in the supernatant from the primary amount of small molecules, as follows in equation 1:

Entrapment efficiency %=M₀−MₛM₀*100 (1),

in which M₀ is the primary concentration of small molecules used in the formulation, and M_{S} is the concentration of non-entrapped small molecules in the supernatant.

L-His crystal controls were prepared using the same procedure, but without the addition of small molecules. Unit cell data for the L-His crystals were collected on a Rigaku Synergy XtaLAB diffractometer. The morphologies of the crystals were observed using a Zeiss 710 Laser Scanning Confocal Microscope (Carl Zeiss Microscopy, Thornwood, N.Y.), an inverted optical microscope (DMTL LED, Leica) connected to a fast camera (MicroLab 3a10, Vision Research), and an SEM (LEO Zeiss 1550 FESEM (Keck SEM) and Zeiss Gemini 500). All SEM images were obtained under high vacuum mode without sputter coating. XRD measurements were performed using a Bruker D8 Advance ECO powder diffractometer (MA) operated at 40 kV and 30 mA (Cu Kα radiation). The crystals were scanned at room temperature from 2θ=10-60° under continuous scanning in 0.02 steps of 2θ min⁻¹.

### Synthesis of Thiolated HA (SH-HA)

Sodium hyaluronate (>43% Glucuronic Acid, Bulk Supplements, Henderson, Nev., USA) was used after being dialyzed against distilled water, followed by lyophilization. L-cysteine methyl ester was synthesized to protect the carboxyl groups of L-cysteine using a previously described method (Rajesh et al., “A Simple and Efficient Diastereoselective Strecker Synthesis of Optically Pure α-Arylglycines,” Tetrahedron 55(37):11295-11308 (1999), which is hereby incorporated by reference in its entirety). The covalent attachment of L-cysteine methyl ester to sodium hyaluronate was achieved through the formation of amide bonds between the primary amino groups of the cysteine methyl ester and the carboxylic groups of hyaluronate. SH-HA was synthesized as described in Ouasti et al., “Network Connectivity, Mechanical Properties and Cell Adhesion for Hyaluronic Acid/PEG Hydrogels,” Biomaterials. 32(27):6456-6470 (2011), which is hereby incorporated by reference in its entirety. Briefly, sodium hyaluronate (2.5 mmol) was dissolved in 100 mL of distilled water, to which N-(3-Dimethylaminopropyl)-N′-ethylcarbodiimide hydrochloride (EDC) (0.5 mmol, >98%, Sigma-Aldrich) and cysteine methyl ester (2.5 mmol) were added under slow stirring. The pH was adjusted to 5.3 by the addition of 1 M NaOH. After incubating the solution for 5 hours, the solution was transferred to dialysis membrane discs (MWCO 3.5 kDa, Thermo Scientific) and dialyzed three-times against 1% NaCl for three days, and finally against distilled water for one day. The solutions were then freeze dried to obtain a white solid and investigated by FTIR in the region from 4000 to 400 cm−1 (120 scans, resolution of 2 cm−1) using an IRAffinity-1S FTIR spectrophotometer (Shimadzu Scientific Instruments/Marlborough, Mass.).

### Synthesis of Thiolated Histidine Methyl Ester (SH-HME)

Histidine methyl ester (HME) was synthesized using as described in Rajesh et al., “A Simple and Efficient Diastereoselective Strecker Synthesis of Optically Pure α-Arylglycines,” Tetrahedron 55(37):11295-11308 (1999), which is hereby incorporated by reference in its entirety. The SH-HME was synthesized by reacting HME (1 mmol) with 2-iminothiolane hydrochloride (0.4 mmol, >98%, Sigma-Aldrich) in PBS (50 mL; pH 7.4) for 12 hours at room temperature. After washing the SH-HME using deionized water, the solution was lyophilized to obtain a powder of SH-HME.

Synthesis of HAase-Responsive, HA-Modified Histidine Crystals with Entrapped DOX (HA-his Crystals)

After synthesizing the L-His crystals with entrapped DOX, SH-HME (0.01 g) was added to the crystal dispersion, followed by the addition of 200 μL ethanol to start growing the SH-HME crystals on the surface of the L-His crystals to form thiolated histidine crystals (SH-His crystals). The SH-His crystals were incubated at room temperature for 3 hours. Next, SH-HA (0.03 g) was added to the SH-His crystal dispersion, and the pH was adjusted to 8 with 1 M NaOH. Then, 50 μL of chloramine T solution (50 mM in PBS buffer, pH 7.4, >98%, Sigma-Aldrich) was added (Fan et al., “Cationic Liposome-Hyaluronic Acid Hybrid Nanoparticles for Intranasal Vaccination with Subunit Antigens,” J. Control Release 208:121-129 (2015), which is hereby incorporated by reference in its entirety), to induce thiol-mediated conjugation of the SH-HA onto the SH-His crystals. After 1 hour incubation at room temperature, the resulting HA-modified histidine crystals (HA-His crystals) were collected from the falcon tubes by centrifugation at 1000×g for 5 minutes, washed with ethanol, freeze-dried, and stored at 4° C.

### In Vitro Enzyme-Triggered Drug Release of DOX-Loaded HA-his Crystals

HAase-triggered drug release profiles of the DOX-loaded HA-His crystals were monitored using HPLC. The DOX-loaded HA-His crystals were incubated with different concentrations of HAase in an acetate buffer (pH=4.3, 37° C.) for 72 hours. To measure the drug release profiles of DOX, HPLC was used to attain data at predetermined time points after incubating the DOX-loaded HA-His crystals with acetate buffer. Supernatants were used to measure the drug release profiles using a dialysis method. In brief, lyophilized HA-His crystals (5 mg) were dispersed in 1 mL of acetate buffer (pH=4.3, 37° C.) containing different concentrations of HAase (0 U/mL, 1 U/mL, and 10 U/mL). The dispersed HA-His crystals were transferred to Spectra/Por® regenerated cellulose dialysis tubes (molecular weight cutoff=10000, Float A lyzer) immersed in 15 mL of acetate buffer (pH=4.3, 37° C.) containing 1.6% Triton X-100 and gently shaken at 37° C. in a water bath at 100 rpm. The medium was replaced with fresh medium at predetermined time points. The cumulative release of DOX was calculated as follows in equation 2:

Cumulative release (%)=(Mₜ/M_{∞})*100 (2)

in which Mₜ is the amount of DOX released from the crystals at time t, and M_{∞} is the amount of DOX in the crystals.

### Statistical Analysis

The results were subjected to analysis of variance (ANOVA) using SPSS software package version 15.0 for Windows. All measurements were performed in triplicate. Mean comparisons were performed using the post hoc multiple comparison Duncan test to determine if differences were significant at P<0.05.

### Example 1—Preparation of Polymorphic Histidine Crystals

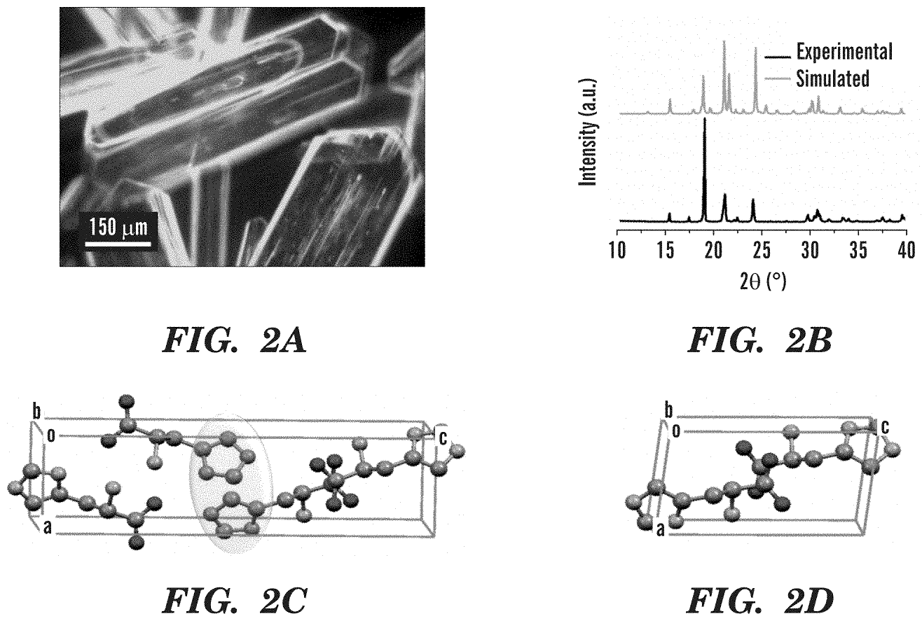

In addition to its well-known roles as an electrophilic acid, L-His features two nitrogen atoms, designated as Nδ1 and Nε2, in its heterocyclic imidazole system, which serve as hydrogen bond acceptor and hydrogen bond donor, respectively (Warzajtis et al., “Mononuclear Gold(III) Complexes with L-Histidine-Containing Dipeptides: Tuning the Structural and Biological Properties by Variation of the N-Terminal Amino Acid and Counter Anion,” Dalton Trans. 46:2594-2608 (2017), which is hereby incorporated by reference in its entirety). Anti-solvent crystallization was performed to synthesize L-His crystals, adding ethanol as the antisolvent to an aqueous solution of L-His at a 1:1 volume ratio (FIG. 2A). The size of the crystals can be tuned from the sub-micron to micron scale, depending on the crystal growth time and antisolvent (Roelands et al., “Antisolvent Crystallization of the Polymorphs of L-Histidine as a Function of Supersaturation Ratio and of Solvent Composition,” Crystal Growth & Design 6(4):955-963 (2006), which is hereby incorporated by reference in its entirety). The L-His crystals display bright emission at 500 nm (405 nm excitation), which we attribute to suppressed nonradiative decay by intramolecular motion due to the close molecular packing of the crystal.

### Example 2—X-Ray Diffraction (XRD) Pattern of L-Histidine (L-His) Crystals

The diffraction peaks of the L-His crystal was in good agreement with the simulated diffraction peaks of the crystal from the Cambridge Crystallographic Data Center (CCDC, CIF code 1206541) (FIG. 2B). The unit cell data of the resulting pure L-His crystals was measured and found to be consistent with a previous study of L-His by Madden et al., “The Crystal Structure of Orthorhombic Form of L-(+)-Histidine,” Acta. Crysta. B28:2377-2382 (1972), which is hereby incorporated by reference in its entirety (CIF code 1206541) (FIG. 2C). X-ray crystallography of the L-His crystals showed a mixture of the stable polymorph A with the orthorhombic space group P212121 and Z=4 molecules in the unit cell, and the metastable polymorph B with the majority being polymorph A. The relative fractions of these polymorphs can be tuned by changing the supersaturation ratio of L-His in aqueous solution (Roelands et al., “Antisolvent Crystallization of the Polymorphs of L-Histidine as a Function of Supersaturation Ratio and of Solvent Composition,” Crystal Growth & Design 6(4):955-963 (2006) and Wantha et al., “Effect of Ethanol on Crystallization of the Polymorphs of L-Histidine,” J. Crystal Growth 490:65-70 (2018), which are hereby incorporated by reference in their entirety). When the L-His molecules arrange in the stable polymorph A crystals, they orient imidazole rings in the vicinity of each other, creating a hydrophobic domain within the structure (FIG. 2C).

### Example 3—Entrapment of Small Hydrophobic Compounds within L-his Crystals

Since the structure of the L-His crystals therefore features several hydrophobic interior domains while displaying a hydrophilic exterior, applicant sought to determine whether such hydrophobic domains could entrap small molecules with a high entrapment efficiency, three different hydrophobic guest compounds were selected as fluorescent probes (Nile red, pyrene, and β-carotene) and two hydrophilic compounds (fluorescein isothiocyanate (FITC) and norbixin) were selected for comparison. The small molecules were individually added to aqueous solutions of L-His, and subsequently mixed with ethanol. The resulting L-His crystals were collected after 3 hours. X-ray crystallography of the L-His crystals loaded with small molecules showed the change of crystal's space group from orthorhombic space group P212121 (Z=4) to the monoclinic space group P21 (Z=2) in the unit cell (FIG. 2D).

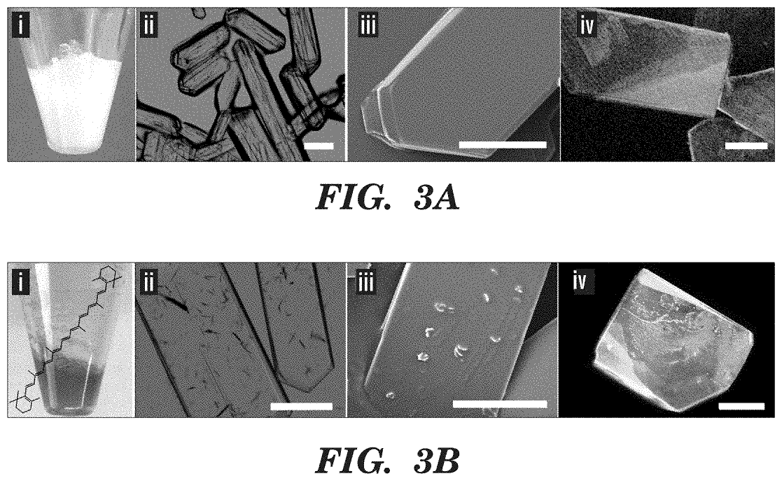

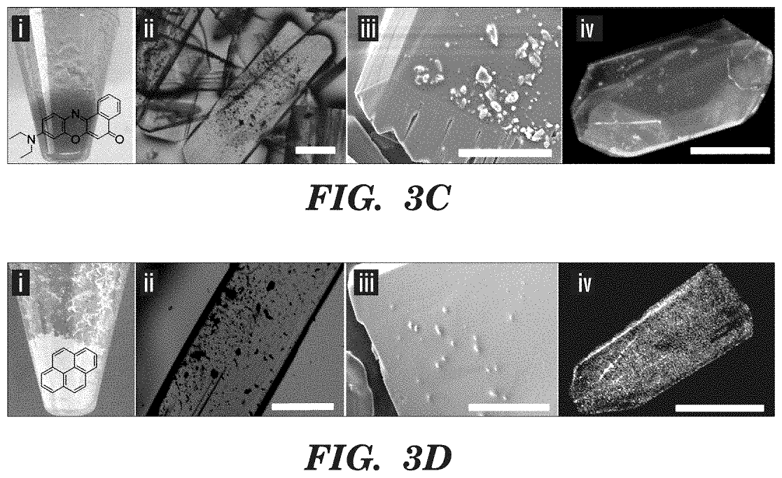

Crystals were also observed using optical, scanning electron (SEM), and confocal laser scanning microscopy (CLSM; FIGS. 3A-3D). The hydrophilic small molecules (FITC and norbixin) were not observed entrapped inside the L-His crystals, instead remaining in solution. However, fluorescence by the hydrophobic β-carotene, Nile red, and pyrene compounds was observed inside the crystals (FIGS. 3A-3D, iv). These observations demonstrate the entrapment of β-carotene, Nile red, and pyrene within the L-His crystals with entrapment efficiencies of ˜96%, 62%, and 87%, respectively, as determined using high-performance liquid chromatography (HPLC). These results indicate that the L-His crystals are specific for the entrapment of hydrophobic small molecules. The inclusion of such hydrophobic small molecules inside the L-His crystals may be noncovalent in nature, driven by hydrophobic interactions, hydrogen bonding, and π-π stacking (Chen et al., “Noncovalent Sidewall Functionalization of Single-Walled Carbon Nanotubes for Protein Immobilization,” J. Am. Chem. Soc. 123(16):3838-3839 (2001) and Liu et al., “Supramolecular Chemistry on Water-Soluble Carbon Nanotubes for Drug Loading and Delivery,” ACS Nano. 1(1):50-56 (2007), which are hereby incorporated by reference in their entirety) between the imidazole rings of the L-His molecules and the aromatic regions and/or double bonds of the hydrophobic small molecules. The entrapment efficiency may depend on the molecular structure of the small molecules and their ability to fit inside the L-His crystal structure.

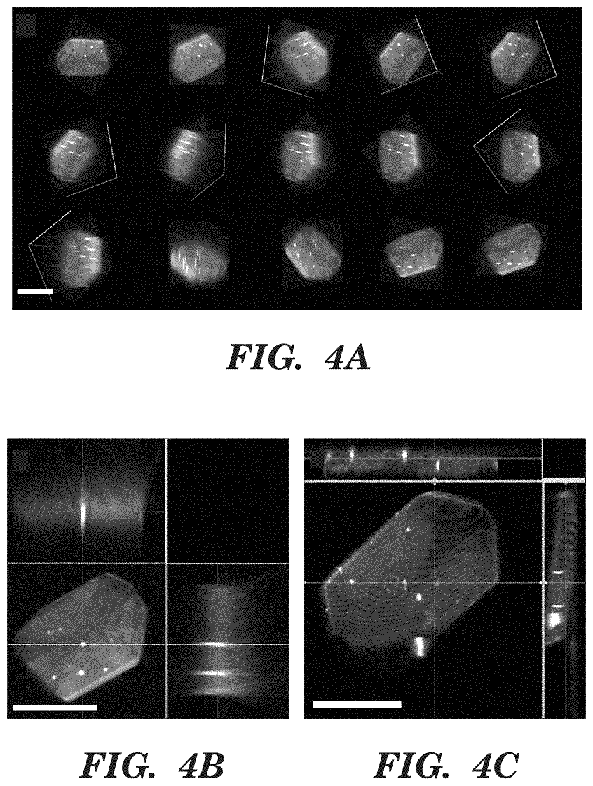

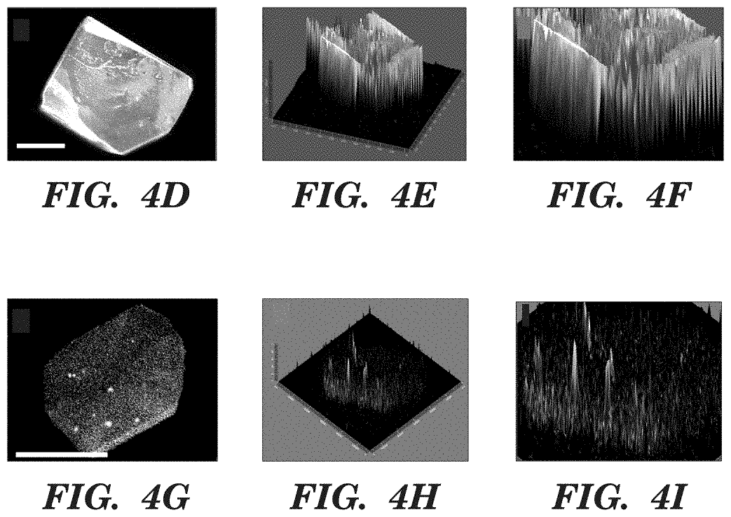

The CLSM imaging results of the loaded L-His crystals along the z optical axis (z-stack) indicates that the localization of the hydrophobic small molecules occurs at the central plane of focus (FIGS. 4A-4I). FIG. 4 demonstrates the entrapment of hydrophobic Nile red (FIGS. 4A-4B) and pyrene (FIG. 4C) inside the L-His crystals from different dimensional perspectives. FIGS. 4D-4I verify that the fluorescent signal of the β-carotene (FIGS. 4D-4F) and Nile red (FIGS. 4G-4I) is indeed localized within the structure of the L-His crystals. The entrapment of small molecules inside the fluorescent L-His crystals not only offers the whole system a hydrophilic surface, which can address the challenges of poor solubility and distribution of hydrophobic small molecules in biological systems, but also provides protection and controlled release of the entrapped small molecules.

### Example 4—X-Ray Diffraction Pattern of L-his Crystals Loaded with Small Molecules

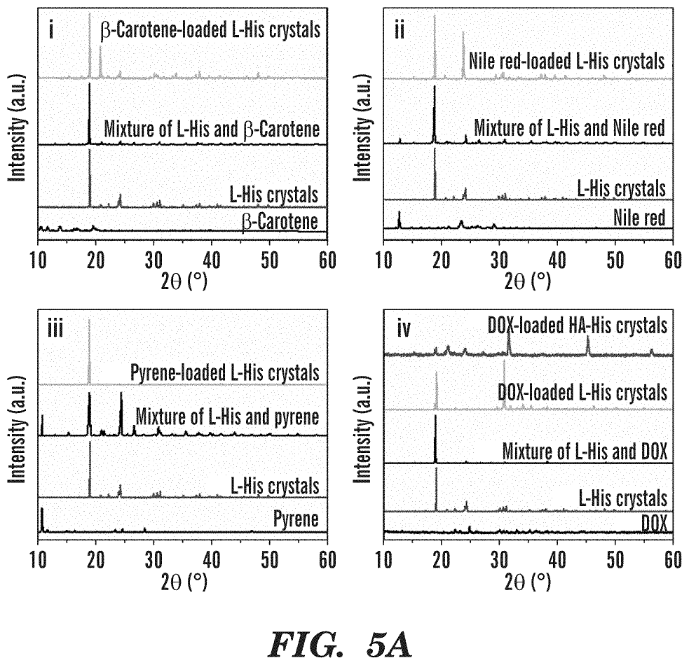

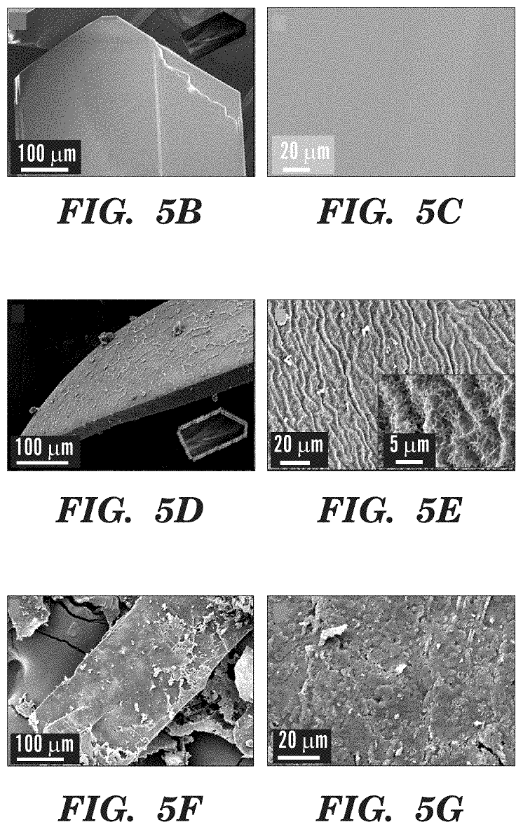

FIG. 5A illustrates the XRD patterns of the pure small molecules (FIG. 5A), pure L-His crystals (FIG. 5A), a dry mixture made of the L-His crystals with the powders of the various small molecules (FIG. 5A), and the small molecule-loaded L-His crystals (FIG. 5A). A characteristic powder diffraction peak of polymorph A appears at 20-19° (FIG. 5A). The XRD analysis of crystals obtained from small molecule-loaded L-His crystals (FIG. 5A) yields a different XRD pattern in comparison with the pure L-His crystals (FIG. 5A). The XRD patterns of the L-His crystals loaded with β-carotene and Nile red show an increase in the intensity of the peaks at 2θ˜220 and 24°, respectively, while the XRD pattern of the pyrene-loaded L-His crystals remains similar to the pure L-His crystals (FIG. 5A).

The changes in the peak intensities indicate the change of electron density inside the unit cell and where the atoms are located (Guo et al., “Loading of Ionic Compounds into Metal-Organic Frameworks: A Joint Theoretical and Experimental Study for the Case of La³,” Phys. Chem. Chem. Phys. 16(33):17918-17923 (2014), which is hereby incorporated by reference in its entirety), and can be influenced by the inclusion of hydrophobic small molecules. This result is in good agreement with the results of single crystal X-ray crystallography, showing the change of L-His crystals' unit cell upon the loading of small molecules (FIGS. 2C-2D). The dominant peaks of the pure small molecules at 20-19°, 13°, and 12° for β-carotene, Nile red, and pyrene, respectively (black lines), disappear in the small molecule-loaded crystal samples (green lines), which confirms the loading of the small molecules inside the structure of the L-His crystals. In contrast, for the manual dry mixture of the L-His crystals and small molecules (blue lines), the XRD patterns are different and the dominant peaks of the small molecules at 2θ˜19°, 13°, and 12° for β-carotene, Nile red, and pyrene remain (FIG. 5A, i-iv).

### Example 5—Doxorubicin (DOX)-Loaded L-Histidine Crystals

Due to the exceptional ability of L-His crystals to fluoresce and entrap hydrophobic small molecules within its hydrophilic structure, applicant investigated whether L-His crystals could be used to entrap doxorubicin, a highly hydrophobic chemotherapeutic, to address its poor solubility, which can cause cardiotoxicity and lowered systemic bioavailability (Torchilin VP, “Targeted Polymeric Micelles for Delivery of Poorly Soluble Drugs,” Cell Mol. Life Sci. 61(19-20):2549-2559 (2004), which is hereby incorporated by reference in its entirety).

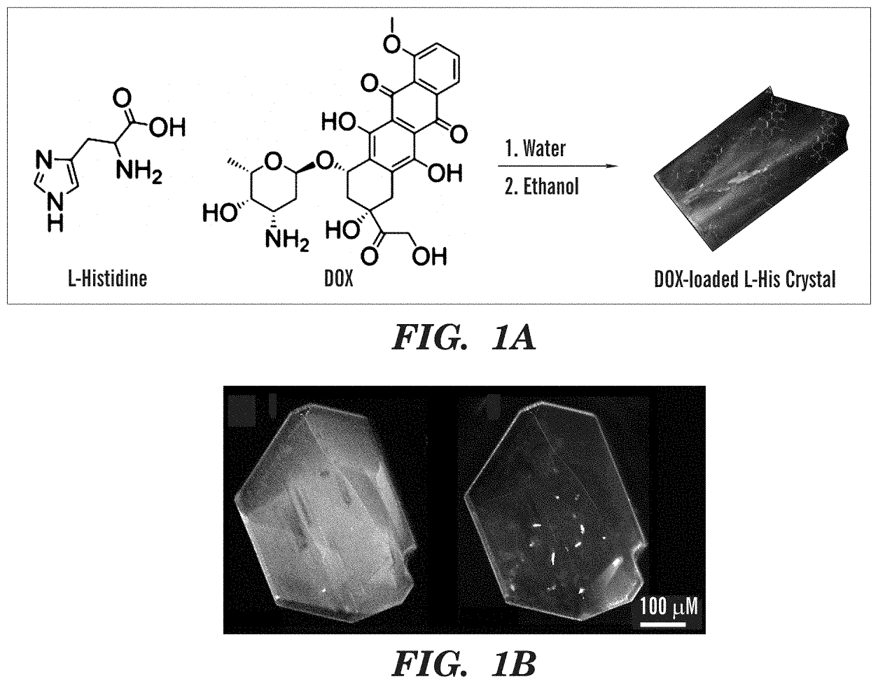

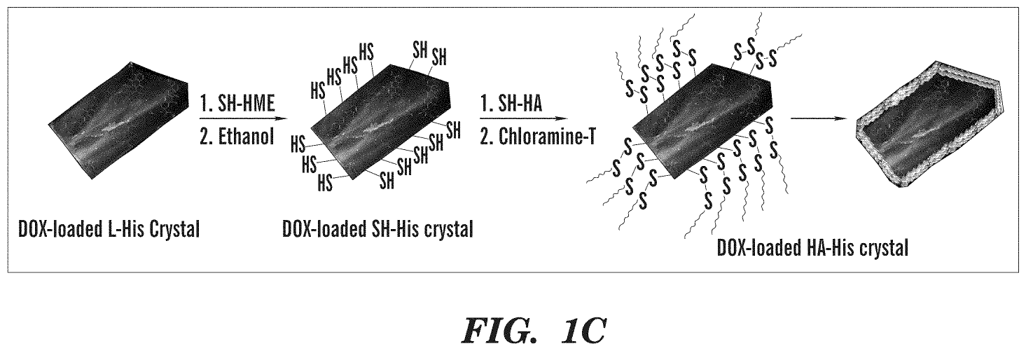

FIG. 1B shows the L-His crystals loaded with DOX, featuring an entrapment efficiency of 55%. The XRD patterns of the L-His crystals loaded with DOX show an increase in the intensity of the peak at 20-320 (green line), indicating the change of electron density inside the unit cell is potentially influenced by the inclusion of DOX molecules (FIG. 5A, iv).

### Example 6—Modification of the Surface of L-his Crystals for Targeted Drug Delivery

Applicant demonstrates that the surface of L-His crystals can be chemically modified to make them site-specific for targeted drug delivery to a specific site of action. The surface of DOX-loaded L-His crystals was chemically modified using hyaluronic acid (HA) (FIG. 1C). HA is a natural, non-toxic and biodegradable acidic polysaccharide composed of N-acetylglucosamine and D-glucuronic acid disaccharide units (Lee et al., “Target-Specific Gene Silencing of Layer-by-Layer Assembled Gold-Cysteamine/siRNA/PEI/HA Nanocomplex,” ACS Nano. 5(8):6138-6147 (2011), which is hereby incorporated by reference in its entirety). HA can serve as an active targeting ligand with high binding affinity to cell-membrane-bound CD44 receptors (Zhu et al., “Drug Delivery: Tumor-Specific Self-Degradable Nanogels as Potential Carriers for Systemic Delivery of Anticancer Proteins,” Adv. Funct. Mater. 28(17):1707371 (2018), which is hereby incorporated by reference in its entirety) which are found on the surface of several malignant tumor cells (Wang et al., “CD44 Antibody-Targeted Liposomal Nanoparticles for Molecular Imaging and Therapy of Hepatocellular Carcinoma,” Biomaterials. 33(20):5107-5114 (2012); Li et al., “Redox-Sensitive Micelles Self-Assembled from Amphiphilic Hyaluronic Acid-Deoxycholic Acid Conjugates for Targeted Intracellular Delivery of Paclitaxel,” Biomaterials. 33(7):2310-20 (2012); and Jiang et al., “Dual-Functional Liposomes Based on pH-Responsive Cell-Penetrating Peptide and Hyaluronic Acid for Tumor-Targeted Anticancer Drug Delivery,” Biomaterials. 33(36):9246-9258 (2012), which are hereby incorporated by reference in their entirety).

Applicant investigated whether L-His crystals could be modified with HA to enhance the specificity of the L-His crystals to deliver DOX to tumor cells and decrease the chance of cytotoxicity and the drug's uptake by normal cells. More importantly, HAase, which plays a significant role in tumor growth, invasion, and metastasis, is widely distributed in the acidic tumor matrix and cleaves internal β-N-acetyl-D-glucosamine linkages in the HA (Jiang et al., “Dual-Functional Liposomes Based on pH-Responsive Cell-Penetrating Peptide and Hyaluronic Acid for Tumor-Targeted Anticancer Drug Delivery,” Biomaterials. 33(36):9246-9258 (2012), which is hereby incorporated by reference in its entirety). HAase is increased in various malignant tumors, including head and neck, colorectal, brain, prostate, bladder, and metastatic breast cancers (Choi et al., “Smart Nanocarrier Based on PEGylated Hyaluronic Acid for Cancer Therapy,” ACS Nano. 5(11):8591-8599 (2011), which is hereby incorporated by reference in its entirety). HA binds to the receptor (CD44) on the surface of the cancer cell and is then cleaved by HAase (Choi et al., “Smart Nanocarrier Based on PEGylated Hyaluronic Acid for Cancer Therapy,” ACS Nano. 5(11):8591-8599 (2011), which is hereby incorporated by reference in its entirety). Applicant hypothesized that this enzyme could be used to hydrolyze HA on the surface of HA-His crystals, allowing the L-His crystals to dissolve in the aqueous matrix and efficiently release the entrapped DOX.

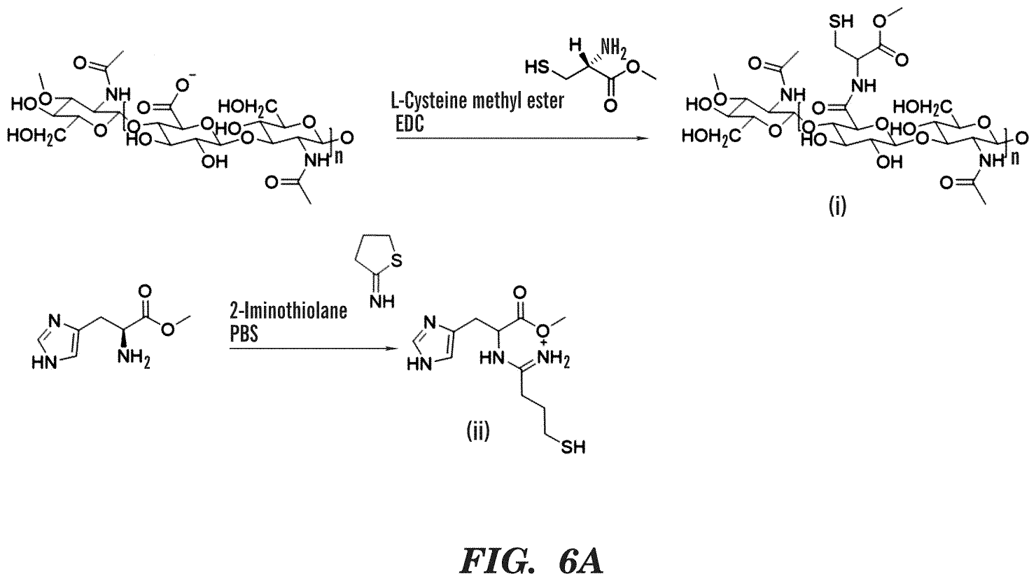

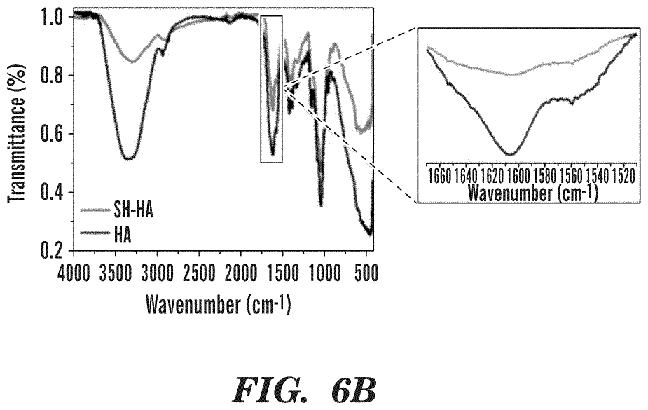

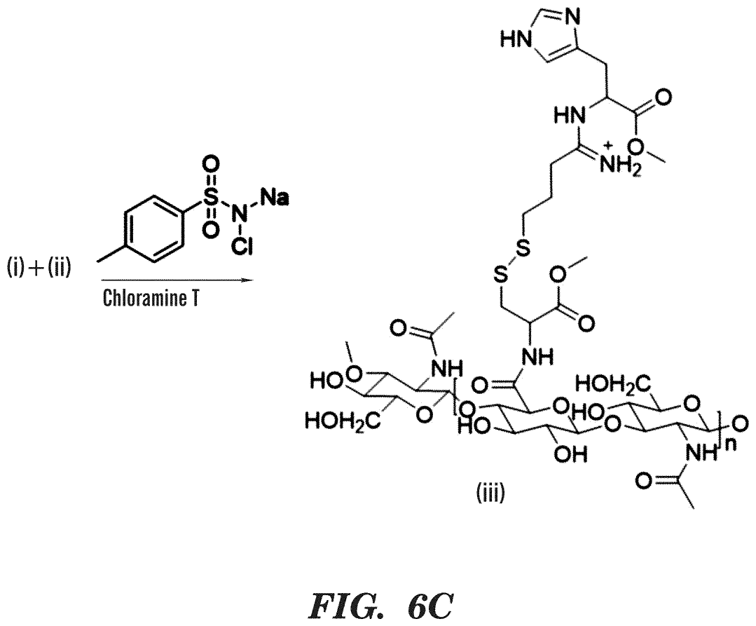

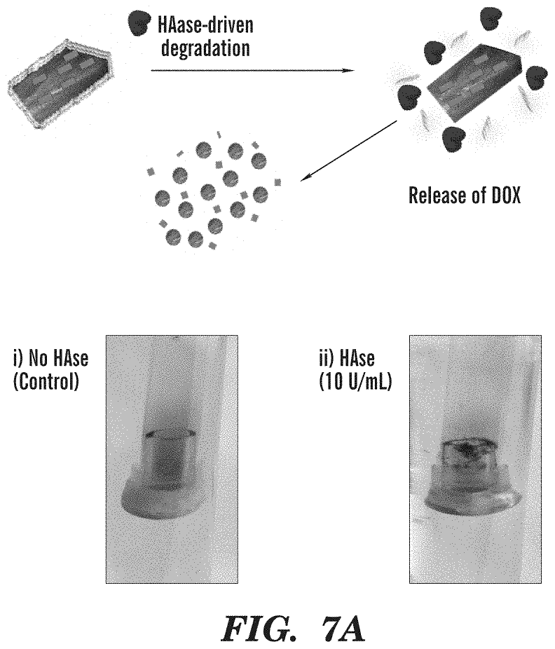

To modify the surface of L-His crystals with HA, the surface of the L-His crystals was first modified with thiolated histidine methyl ester (SH-HME), and then cross-linked the SH-HME with thiolated hyaluronic acid (SH-HA) through the formation of disulfide bonds (FIG. 1C). FIG. 6A shows the schematic illustration for the synthesis of SH-HA, SH-HME. The comparison between Fourier transform infrared (FTIR) spectra of HA and SH-HA shows a significant decrease of the peak at 1610-1620 cm−1 associated with the HA carboxyl groups, confirming the formation of SH-HA (FIG. 6B). FIG. 6C shows the formation of disulfide bonds between SH-HA and SH-HME. The L-His crystals are smooth before surface modification (SEM images, FIGS. 5B-5C). The chemical modification of the L-His crystals through the formation of disulfide bonds between SH-HME and SH-HA forms a uniform layer of HA on the surface of the L-His crystals (FIGS. 5D-5E). In contrast, applying HA solution directly to the surface of the L-His crystals does not result in a uniform layer on the crystal (FIGS. 5F-5G). Surface modification of the L-His crystals with HA also changes the XRD pattern, showing two dominant peaks at 20-330 and 460 (FIG. 5A, iv).

### Example 7—DOX is Released from HA-Crystals Following Incubation With HAase

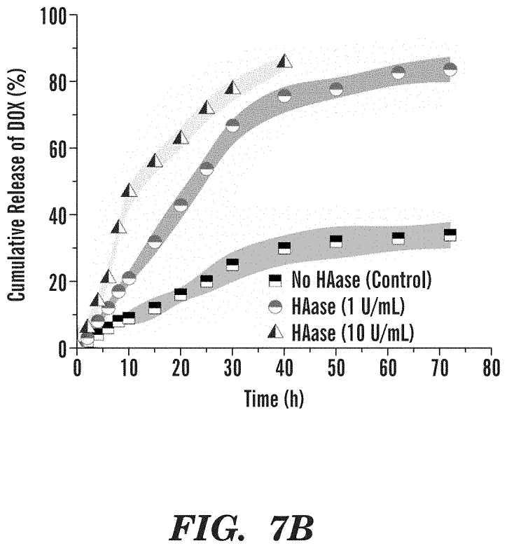

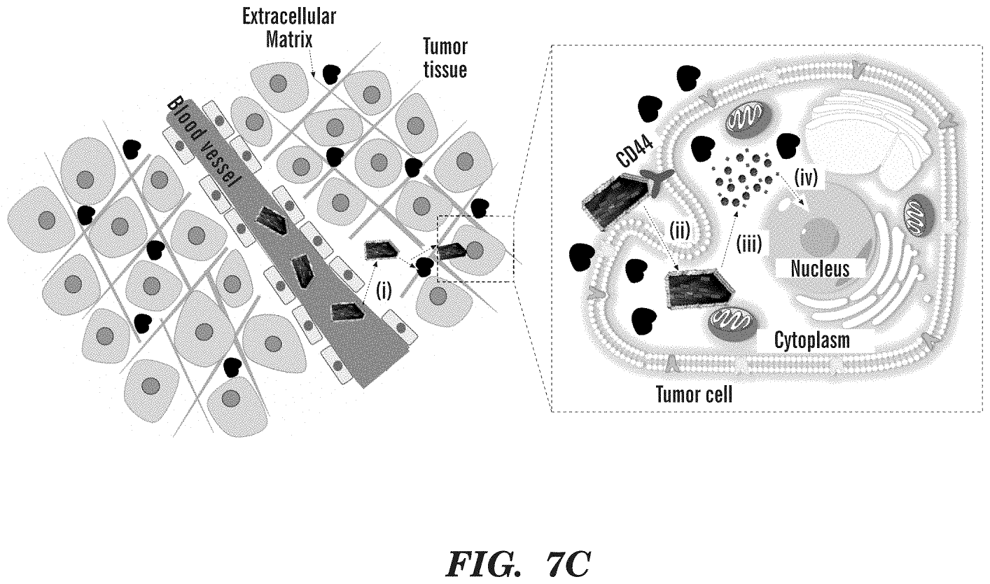

FIG. 7A illustrates how HA-His crystals start to disintegrate in the presence of HAase after 4 hours. In vitro release experiments revealed that less than 35% of DOX is released from the HA-His crystals after 72 hours in phosphate buffer, whereas 84% of DOX is released during that same time in the presence of 1 U/mL HAase (FIG. 7B). In the presence of 10 U/mL HAase, the release rate is accelerated and 86% of DOX is released in 40 hours (FIG. 7B). This result indicates that the HA-His crystals incubated with HAase markedly increase the release of DOX. Thus, HA-His crystals can potentially bind to CD44 receptors on the surface of tumor cells, enhancing the cellular uptake, and then release entrapped DOX upon degradation by HAase to the intracellular compartments of tumors, increasing apoptosis of tumor cells (FIG. 7C).

### Discussion of Examples 1-7

The results presented herein demonstrate the entrapment of hydrophobic small molecules inside the hydrophobic domains of L-His crystals, providing a biocompatible platform for protecting hydrophobic drugs. Since the entrapment of hydrophobic small molecules is at the molecular level, the entrapment efficiency is relatively high and possibly depends on the molecular structure of the small molecules. The modification of the L-His crystals at the surface using polymers and/or hydrogels could enable intracellular trafficking and site-specific delivery of hydrophobic therapeutics, providing a drug-delivery system with targeting features. For example, the L-His crystals with HA covalently bonded to their surface and loaded with DOX are able to target tumor cells and control the release of DOX in response to HAase overexpressed in these cells. The composition of the surface can be controlled and tuned for optimization with other enzymes and physiological media. Releasing the entrapped hydrophobic drugs as the HA-His crystals are degraded and dissolved in the aqueous media can also reduce the chance of local toxicity to normal cells due to drug aggregation. The successful entrapment and targeted release of hydrophobic small molecules in HA-His crystals suggests further study is warranted to probe the possible implementation of amino acid crystals in promoting the delivery of hydrophobic therapeutics with low solubility and/or delivery of a combination of hydrophobic drugs to treat multidrug resistance. This strategy helps to address issues related to the poor solubility and low bioavailability of such molecules. These L-His crystals can also be investigated in terms of improving the imaging and tracking of entrapped therapeutic agents due to the crystals' natural fluorescence properties.

### Materials and Methods for Examples 8-11

### Preparation of the Amino Acid Crystals

Amino acid solutions (30 mg/mL), including L-histidine, L-glutamine, L-isoleucine, L-asparagine, L-valine, L-threonine, and L-methionine (>98%, Sigma-Aldrich) were prepared individually by dissolving the amino acid powder in milli-Q water using a vortex mixer at ambient temperature in a Corning® 15 mL centrifuge tube with a closed cap. Then, 3 mL of 200 proof ethanol (KOPTEC, PA, US) was added to 3 mL of the aqueous solution of amino acid as an antisolvent. The amino acid crystals were collected after 6 hours.

### Characterization

Unit cell data for the amino acid crystals were collected on a Rigaku Synergy XtaLAB diffractometer. Morphologies of the crystals were observed using a Zeiss 710 Laser Scanning Confocal Microscope with a 25×/0.8 NA oil immersion objective (Carl Zeiss Microscopy, Thornwood, N.Y.), an inverted optical microscope (DMIL LED, Leica) connected to a fast camera (MicroLab 3a10, Vision Research), and SEM (JCM-6000 Benchtop scanning electron microscope, software version 2.4 (JEOL Technics Ltd., Tokyo, Japan)). Moreover, the Zeiss 710 confocal microscope was equipped with lasers at 405 nm, 488 nm, 561 nm, and 633 nm, and the spectral detector allows the collection of a series of emission wavelengths with lambda scan mode. XRD measurements were performed using a Bruker D8 Advance ECO powder diffractometer (MA) operated at 40 kV and 30 mA (Cu Kα radiation). The crystals were scanned at room temperature from 20=10-60° under continuous scanning in 0.02 steps of 2θ min⁻¹.

The lifetime of the amino acid crystals was investigated through time-correlated single photon counting fluorescence measurements (TCSPC), which were carried out using ˜120 fs pulses at 800 nm delivered at an 80 MHz repetition rate from a Spectra-Physics Mai-Tai Ti:S laser equipped with DeepSee dispersion compensation. The Ti:S laser was coupled to a Zeiss 880 laser scanning microscope which was used to locate and focus on the crystals. Two-photon generated epi-fluorescence was separated from the excitation using a 670 nm long pass dichroic filter, which directed the emission to a GaAsP photomultiplier tube after passing through a broad blue band-pass filter (BGG22, Chroma Technology Corp, VT). The laser power was attenuated using a near infrared (NIR) Acousto Optic Modulator (AOM) to keep the photon detection rate to less than 0.2% of the repetition rate to avoid photon pile-up. An instrument response function (IRF) was acquired using a Z-cut quart crystal and used for fitting the TCSPC data. Time-correlated photon counts were acquired using a high-resolution TCSPC module (SPC-830, Becker & Hickl GmbH) and fit to a bi-exponential decay curve, convolved with the IRF, using the SPCImage software package (Becker & Hickl GmbH). The NaCl salt crystals were used as a negative control for the lifetime measurements. The weighed mean lifetime was calculated using the following formula:

((a_1×τ_1)+(a_2×τ_2))/((a_1+a_2))

### Example 8—Crystallization-Induced Emission in Amino Acid Crystals

In many cases, luminogens are highly emissive only in dilute solutions but are nonemissive in the solid state (Mei et al., “Aggregation-Induced Emission: Together We Shine, United We Soar!,” Chem. Rev. 115(21):11718-11940 (2015) and Mei et 1, “Aggregation-Induced Emission: The Whole is More Brilliant than the Parts,” Advanced Materials 26(31):5429-5479 (2014), which are hereby incorporated by reference in their entirety) where molecules may experience strong π-π stacking interactions that lead to quenching (Gopikrishna et al., “Monosubstituted Dibenzofulvene-Based Luminogens: Aggregation-Induced Emission Enhancement and Dual-State Emission,” J. Phys. Chem. C 120(46):26556-26568 (2016), which is hereby incorporated by reference in its entirety). In contrast, there are other small molecules that show induced emission in their solid state (Li et al., “Fluorescence of Nonaromatic Organic Systems and Room Temperature Phosphorescence of Organic Luminogens: The Intrinsic Principle and Recent Progress,” Small 14(38):1801560 (2018) and Nishiuchi et al., “Solvent-Induced Crystalline-State Emission and Multichromism of a Bent 71-Surface System Composed of Dibenzocyclooctatetraene Units,” Chemistry—A European Journal 19(13):4110-4116 (2013), which are hereby incorporated by reference in their entirety). In solution, these molecules experience dynamic intramolecular motion that annihilate their excited state nonradiatively. However, in the solid state the molecules cannot pack through a 71-71 stacking process due to the restricted intramolecular motions (Mei et al., “Aggregation-Induced Emission: Together We Shine, United We Soar!,” Chem. Rev. 115(21):11718-11940 (2015) and Mei et 1, “Aggregation-Induced Emission: The Whole is More Brilliant than the Parts,” Advanced Materials 26(31):5429-5479 (2014), which are hereby incorporated by reference in their entirety).

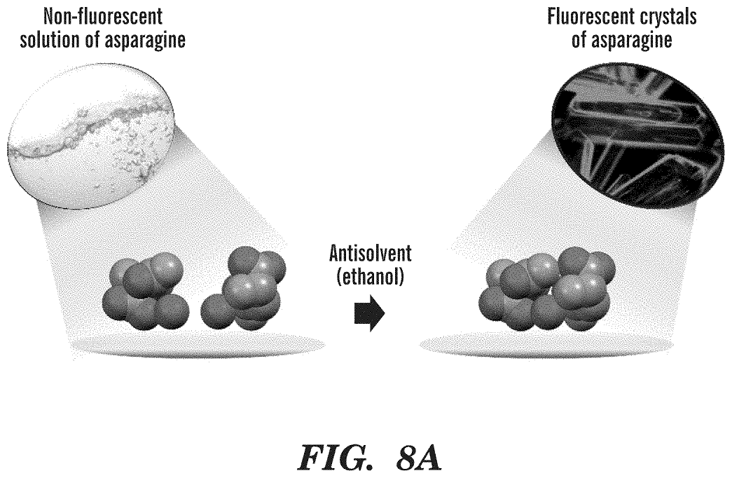

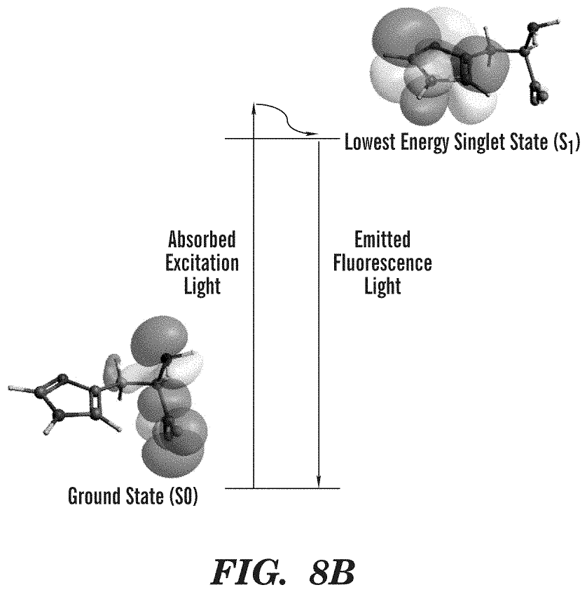

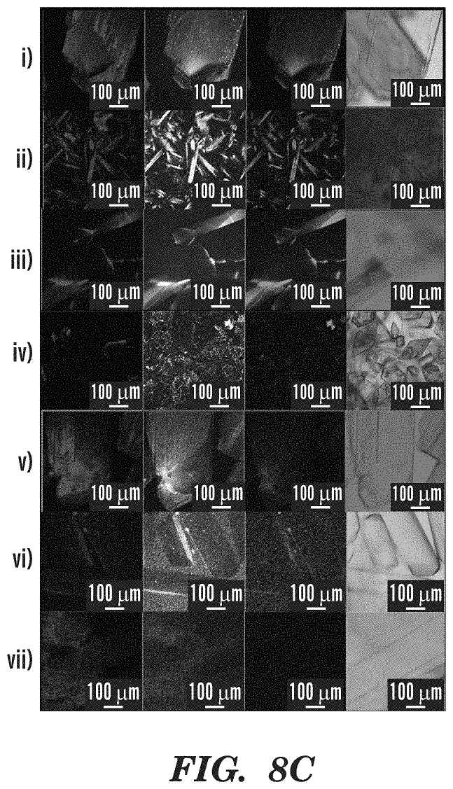

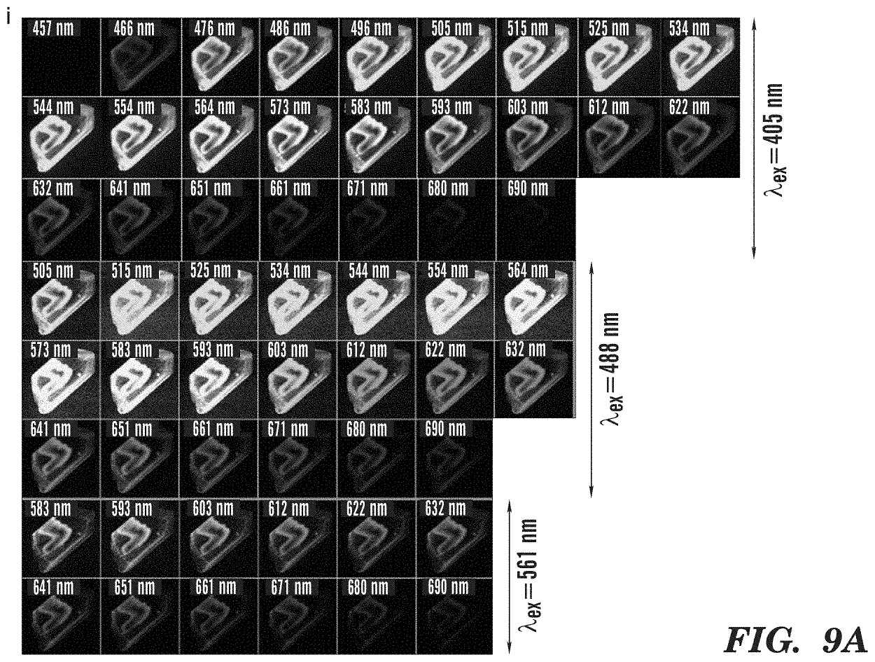

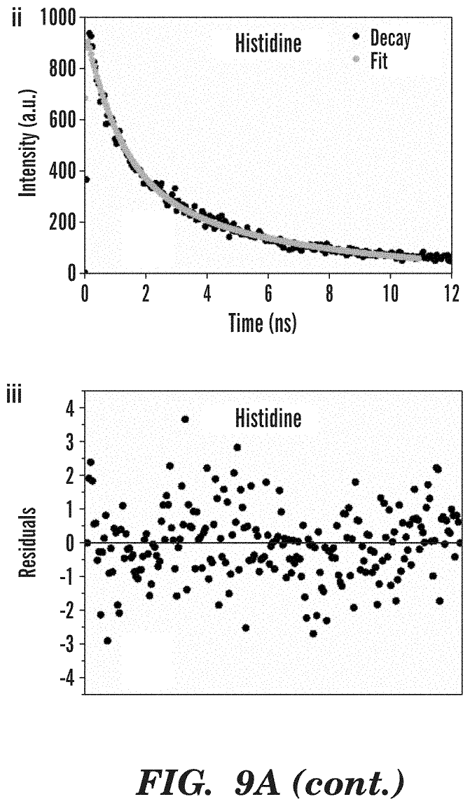

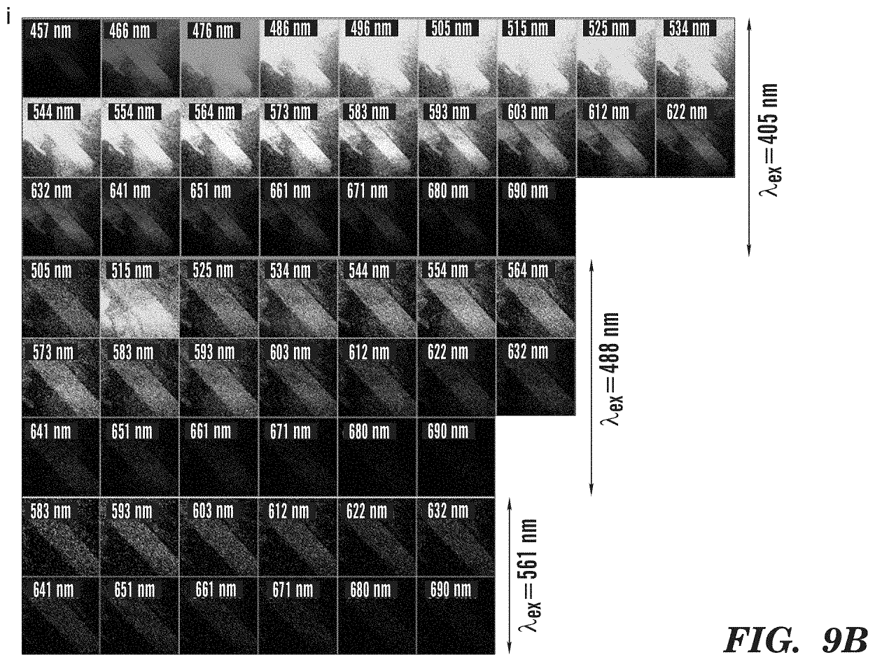

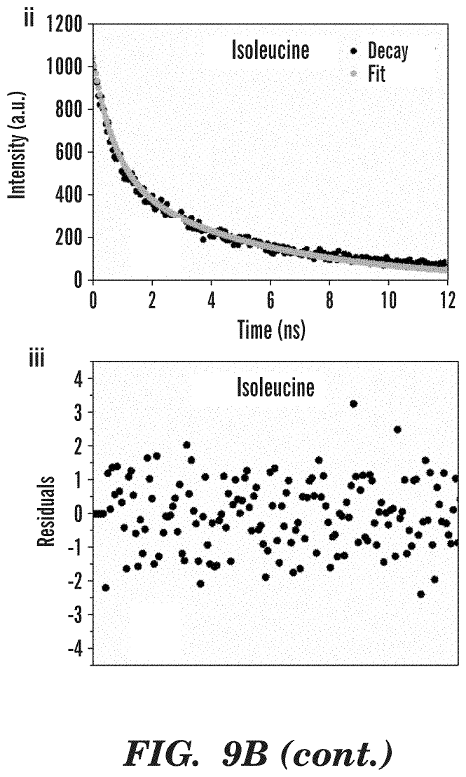

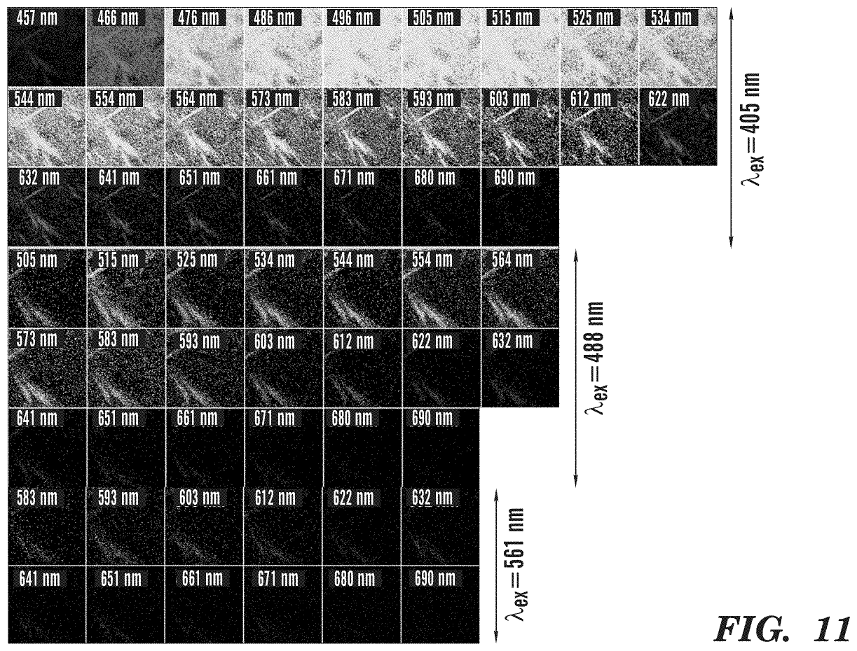

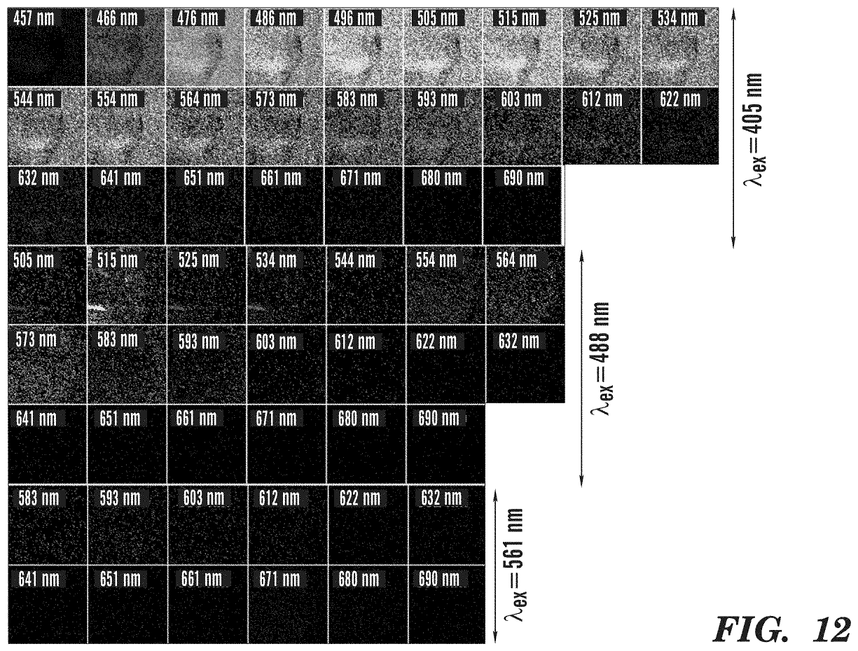

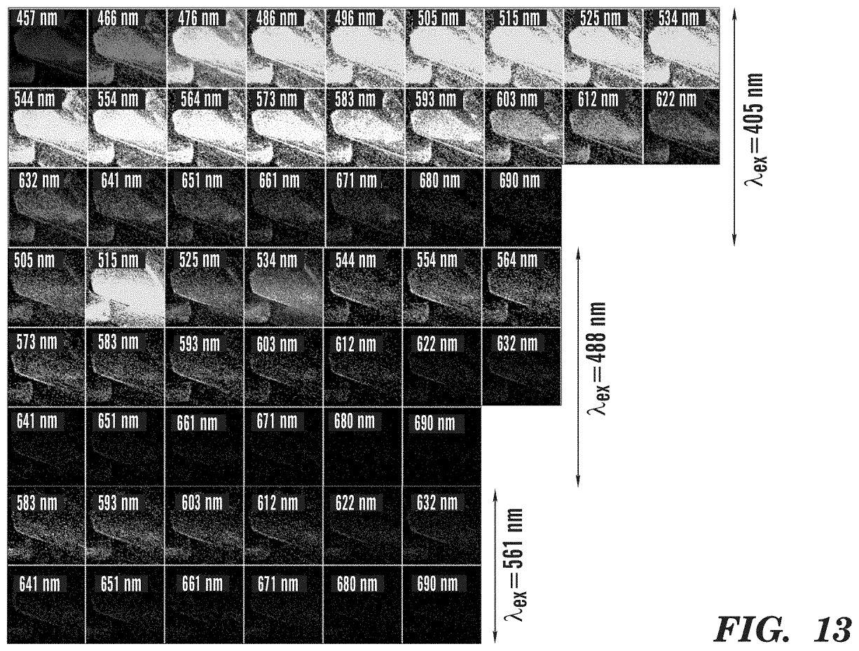

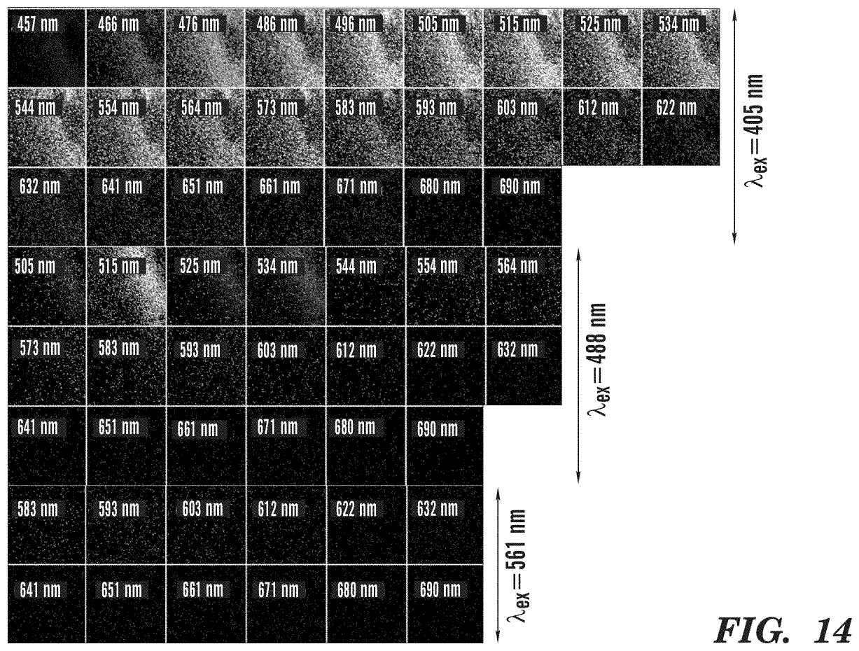

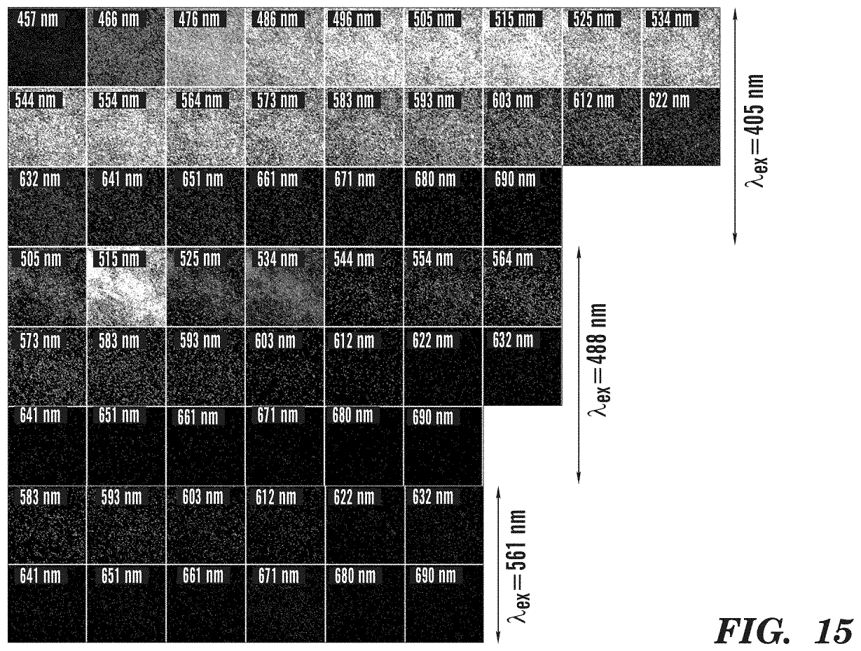

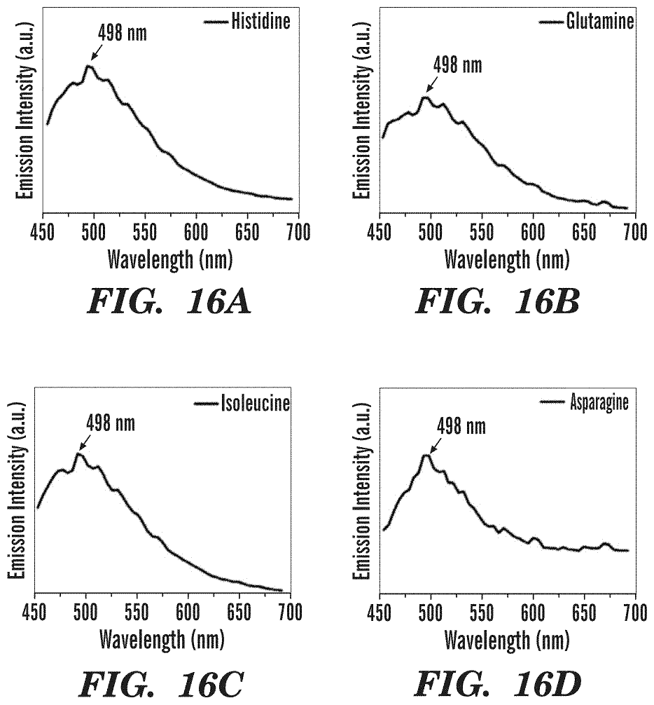

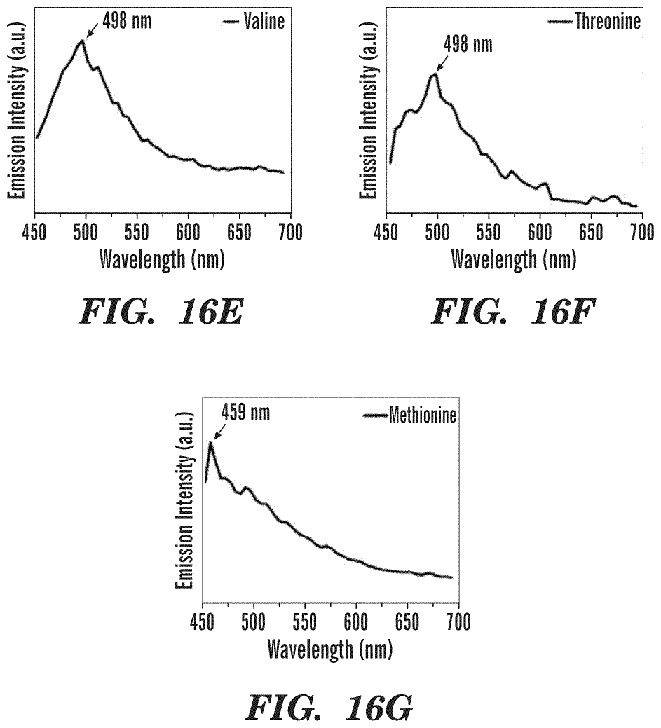

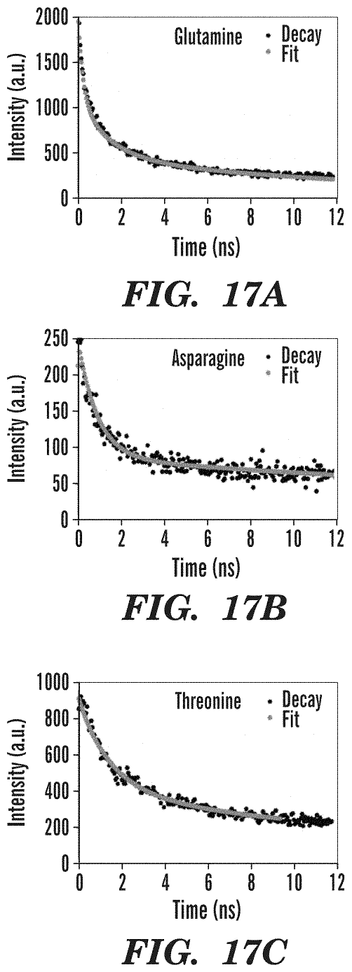

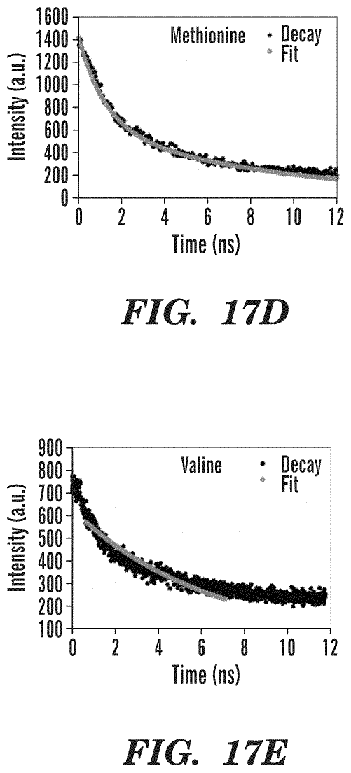

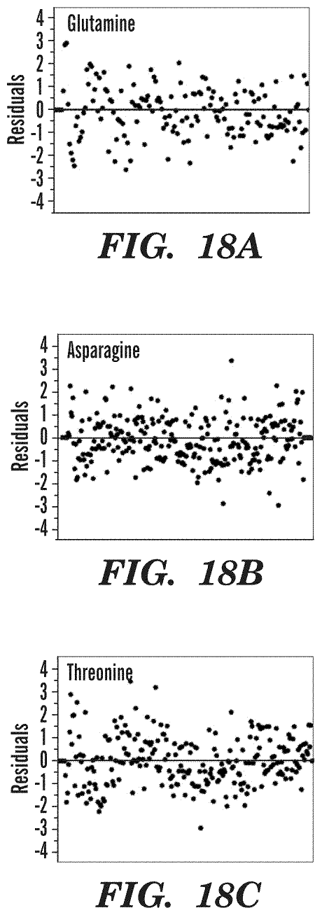

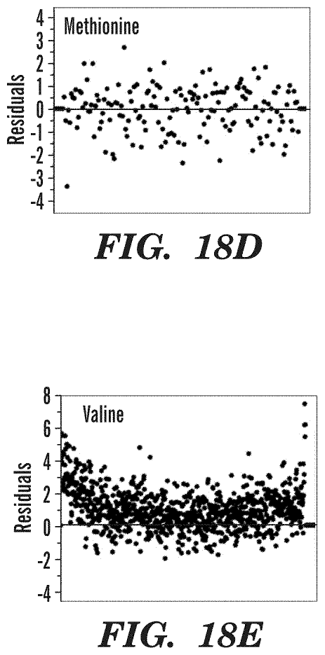

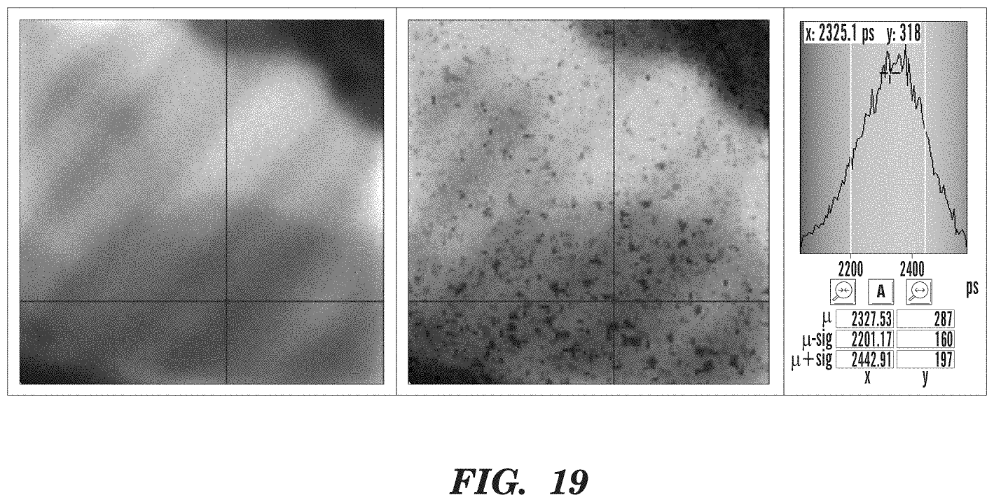

FIGS. 8A-8B demonstrate crystallization-induced emission in amino acid crystals. Crystals of seven amino acids, including L-histidine, L-glutamine, L-isoleucine, L-asparagine, L-valine, L-threonine, and L-methionine were prepared through antisolvent crystallization. Briefly, an aqueous solution of each amino acid was prepared and then ethanol was added as an antisolvent, resulting in the formation of the amino acid crystals (FIG. 8A). Since most of these amino acids are nonaromatic, very little attention has been paid to their photophysical properties in crystalline form. However, it was found that these amino acids have a natural fluorescence emission in their crystalline state that ranges widely from blue to green and red when excited at 405 nm, 488 nm, and 561 nm under confocal laser scanning microscopy (CLSM; FIGS. 8C, 9A-9B (i), and FIGS. 11-15. Of note, none of these amino acids is fluorescent in solution. The amino acid crystals display different fluorescence emission intensities with maximum emission at 498 nm upon excitation at 405 nm, except L-methionine, which features a maximum emission at 459 nm when excited at 405 nm (FIGS. 16A-16G).

### Example 9—Supramolecular Assembly of Amino Acids in the Crystalline Structure

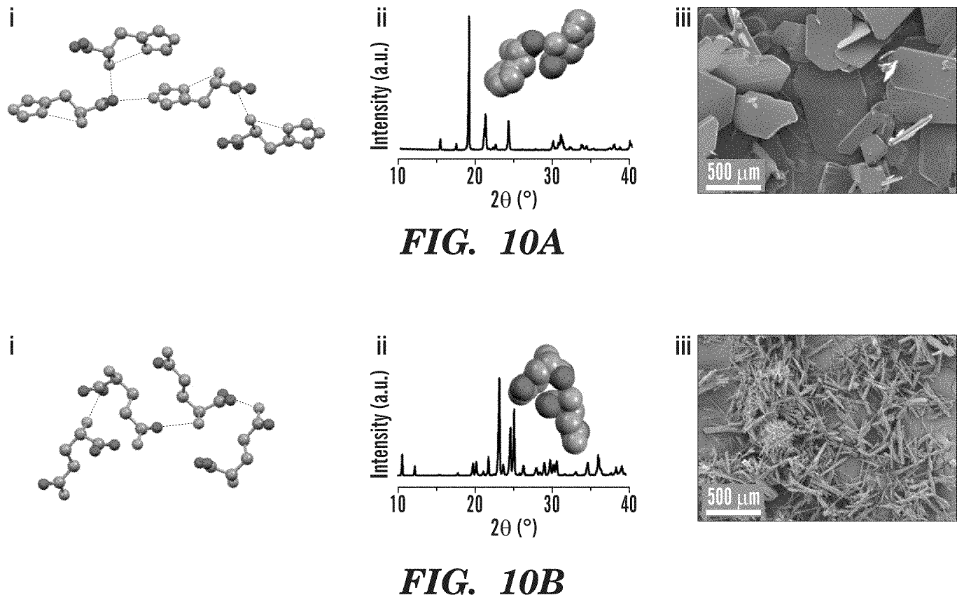

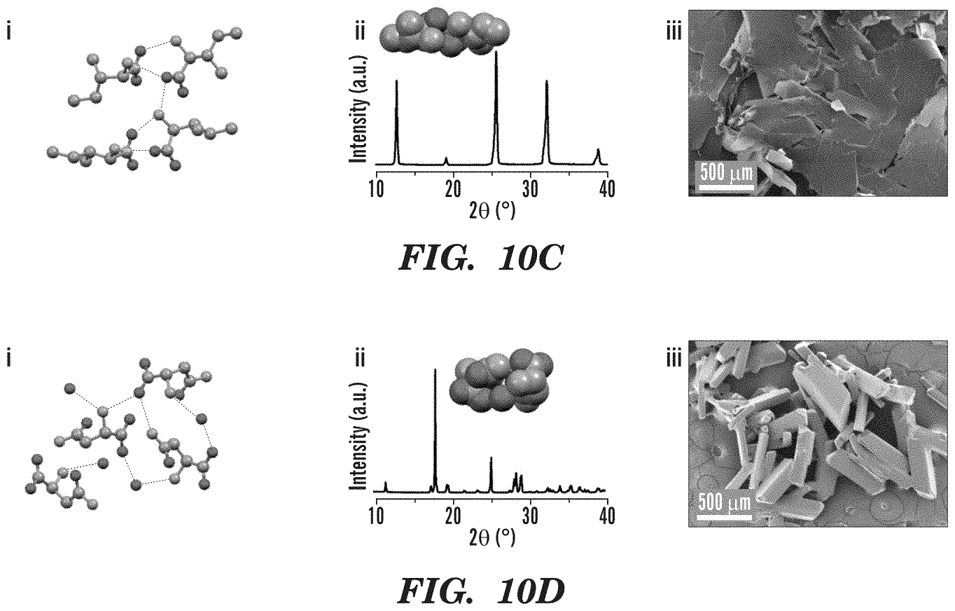

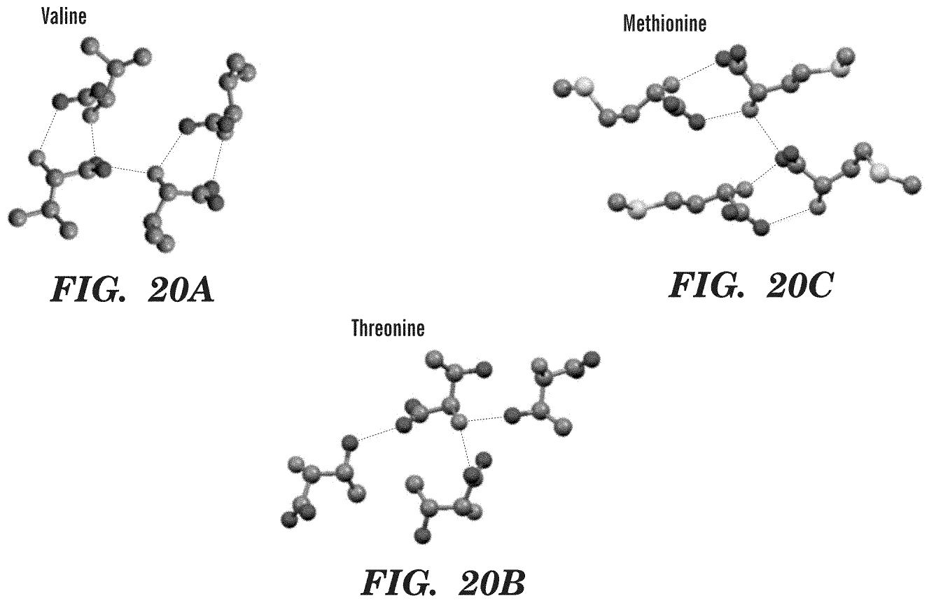

The interplay between chemistry and crystallography is in fact the inter-relationship between the molecular properties and supramolecular assembly of molecules. Therefore, the supramolecular assembly of these amino acids in the crystalline structure was investigated. Applicant determined the structure of amino acid crystals by single crystal X-ray crystallography. The observed unit cell data of crystals were consistent with previous studies of these materials (FIGS. 10A-10D (i) and FIGS. 20A-20C) (Madden et al., “The Crystal Structure of the Orthorhombic Form of L-(+)-Histidine,” Acta Crystallographica Section B 28(8):2377-2382 (1972); Wagner et al., “Charge Density and Topological Analysis of L-Glutamine,” Journal of Molecular Structure 595(1-3):39-46 (2001); Weisinger-Lewin et al., “Reduction in Crystal Symmetry of a Solid Solution: A Neutron Diffraction Study At 15 K of the Host/Guest System Asparagine/Aspartic Acid,” Journal of the American Chemical Society 111(3):1035-1040 (1989); Taratin et al., “Solubility Equilibria and Crystallographic Characterization of the L-Threonine/L-allo-Threonine System, Part 2: Crystallographic Characterization of Solid Solutions in the Threonine Diastereomeric System,” Crystal Growth Design 15(1):137-144 (2014); Gorbitz et al., “L-Isoleucine, Redetermination At 120K,” Acta Crystallographica Section C: Crystal Structure Communications 52(6):1464-1466 (1996); Torii et al., “The Crystal Structure of L-Valine,” Acta Crystallographica Section B: Structural Crystallography 26(9):1317-1326 (1970); and Dalhus et al., “Crystal Structures of Hydrophobic Amino Acids I. Redeterminations of L-Methionine and L-Valine At 120 K,” Acta Chemica Scandinavica 50(6):544-548 (1996), which are hereby incorporated by reference in their entirety). The X-ray crystallography data indicates that the L-histidine, L-glutamine, L-asparagine, and L-threonine crystals are in the orthorhombic P2₁2₁2₁ space group and with Z=4 molecules in the unit cell (CIF codes 1206541, 155068, 1103695, and 1060965, respectively) (FIGS. 10A, 10B, 10D (i) and FIG. 20 B). The crystals of L-isoleucine, L-valine, and L-methionine are in the P2₁ space group and also with Z=4 molecules in the unit cell (CIF codes 126824, 1208817, and 1207980, respectively) (FIG. 10C (i) and FIGS. 20A-20C).

The crystalline structure of the amino acid molecules are formed through the interactions between molecules directed by intermolecular forces (Zhang et al., “Intramolecular Vibrations in Low-Frequency Normal Modes of Amino Acids: L-Alanine in the Neat Solid State,” Acta Chemica Scandinavica 119(12):3008-3022 (2015), which is hereby incorporated by reference in its entirety). The energetic and geometric properties of these intermolecular forces and their influence on the intramolecular forces, however, are much less understood than those of classical chemical bonds (Zhang et al., “Intramolecular Vibrations in Low-Frequency Normal Modes of Amino Acids: L-Alanine in the Neat Solid State,” Acta Chemica Scandinavica 119(12):3008-3022 (2015), which is hereby incorporated by reference in its entirety). One of the strongest interactions is the hydrogen bond, which is holding the organic molecules together in a crystalline structure (Bernstein et al., “Patterns in Hydrogen Bonding: Functionality and Graph Set Analysis in Crystals,” Angewandte Chemie International Edition in English 34(15):1555-1573 (1995), which is hereby incorporated by reference in its entirety). The X-ray crystallography results reveal the hydrogen bonds in the amino acid crystals (FIG. 10A-10D (i), FIGS. 20A-20C). The length and number of hydrogen bonds in the crystal unit cells was measured to compare the density of the hydrogen bonding network for these seven amino acids. Table 1 shows that L-asparagine features the maximum number of hydrogen bonds (8) in its unit cell, while the minimum number of hydrogen bonds (3) was observed for L-threonine and L-glutamine. The length of the hydrogen bonds range from 2.6-3.0 Å (Table 1). So short is this distance that it is very reasonable to assume that such molecular contact is rare in a non-condensed solution state of amino acids.

TABLE 1
Number and Length of Hydrogen Bonds in the Unit Cells of
Amino Acid Crystals.

| Amino acids | Number of H-bonds | Length of H-bonds (Å) |
|---|---|---|
| L-Histidine | 7 | 2.8, 2.8, 2.8, 2.8, 2.8, 2.8, 3.0 |
| L-Glutamine | 3 | 2.8, 2.9, 2.9 |
| L-Isoleucine | 5 | 2.8, 2.8, 2.8, 2.8, 2.8 |
| L-Asparagine | 8 | 2.8, 2.8, 2.8, 2.8, 2.8, 2.8, 2.9, 2.9 |
| L-Valine | 5 | 2.8, 2.8, 2.8, 2.9, 2.9 |
| L-Threonine | 3 | 2.6, 2.8, 3.0 |
| L-Methionine | 5 | 2.8, 2.8, 2.8, 2.9, 2.9 |

### Example 10—Hydrogen Bonding Effects the Fluorescence Emission of Amino Acid Crystals

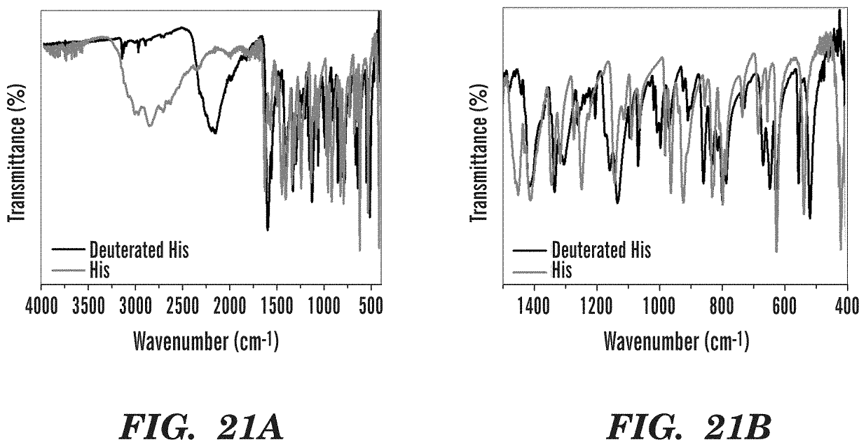

Short-distance interactions in the crystal structure of organic compounds hinder intramolecular motions and vibrations and clearly indicate a definite electronic interaction between the atoms (Mei et al., “Aggregation-Induced Emission: Together We Shine, United We Soar!,” Chem. Rev 115(21):11718-11940 (2015), which is hereby incorporated by reference in its entirety). Thus, the non-radiative energy loss in the excited state is reduced and enhances the photoluminescence character of the organic compound (Mei et al., “Aggregation-Induced Emission: Together We Shine, United We Soar!,” Chem. Rev 115(21):11718-11940 (2015), which is hereby incorporated by reference in its entirety). To confirm the effect of the hydrogen bonding network on the fluorescence emission of the amino acid crystals, deuterated L-histidine crystals were prepared as a model of amino acid incapable of forming hydrogen bonds (FIGS. 21A-21B) and compared the lifetime with the original L-histidine crystals (Table 2). The deuterated L-histidine crystals show a lifetime of 1.95 ns and the original L-histidine crystals show a lifetime of 2.20 ns (Table 2). This change in the fluorescent lifetime indicates that the hydrogen bonding network due to close packing can contribute to the fluorescence emission of these nonaromatic and aromatic amino acids in crystalline form.

TABLE 2
Fluorescence Lifetimes and Weighed
Mean Lifetimes of amino Acid Crystals.

| | | | | | | Mean |
|---|---|---|---|---|---|---|
| I | a₁ (%) | τ₁ (ns) | a₂ (%) | τ₂ (ns) | χ² | lifetime (ns) |
| L-Histidine | 56.51 | 0.67 | 43.48 | 4.19 | 1.32 | 2.20 |
| Deuterated | 65.08 | 0.73 | 34.91 | 4.24 | 1.46 | 1.95 |
| L-Histidine | | | | | | |
| L-Glutamine | 77.93 | 0.85 | 22.06 | 8.49 | 1.18 | 2.53 |
| L-Isoleucine | 59.75 | 0.79 | 40.25 | 5.69 | 1.07 | 2.76 |
| L-Asparagine | 87.39 | 0.9 | 12.61 | 36.40 | 0.97 | 5.38 |
| L-Valine | 9.57 | 5.02 | 90.42 | 6.68 | 2.29 | 6.52 |
| L-Threonine | 61.02 | 1.31 | 21.07 | 19.46 | 1.29 | 4.90 |
| L-Methionine | 66.54 | 1.25 | 33.46 | 9.19 | 1.01 | 3.91 |
| NaCl (Control) | 0.12 | 0.02 | 99.87 | 0.02 | 1.08 | 0.02 |

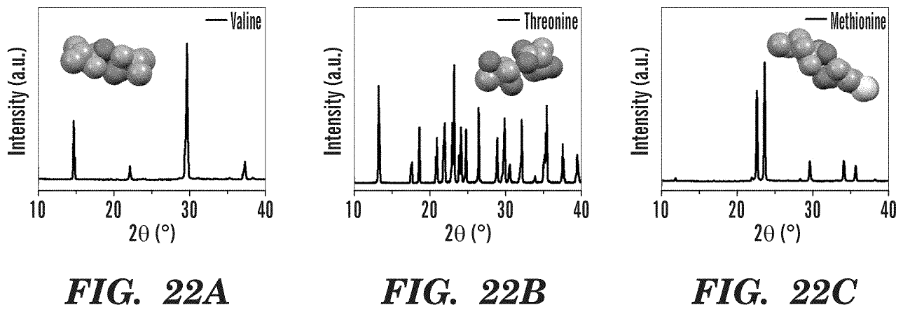

FIGS. 10A-10D (ii) and FIGS. 22A-22C show the X-ray powder diffraction spectra of these seven amino acids in addition to their spacefil models in the crystalline state. The spacefil models also show the molecular packing of the amino acids, highlighting the extremely close contact between the carbonyl and amino moiety of the neighboring molecules (FIG. 10A-10D (ii) and FIG. FIGS. 22A-22C). The n and π electrons of these functional groups can enable electron delocalization between these units due to the effective orbital overlap made possible at the close intermolecular distance (Li et al., “Fluorescence of Nonaromatic Organic Systems and Room Temperature Phosphorescence of Organic Luminogens: The Intrinsic Principle and Recent Progress,” Small 14(38):1801560 (2018) and Wang et al., “Aggregation-Induced Emission of Non-Conjugated Poly (Amido Amine) S: Discovering, Luminescent Mechanism Understanding and Bioapplication,” Chinese Journal of Polymer Science 33(5):680-687 (2015), which are hereby incorporated by reference in their entirety). Such electron delocalization by n-n and n-n coupling in the rigid conformation of nonaromatic systems can allow the suppression of nonradiative processes and stabilization of the excited states in nonaromatic amino acid crystals. Moreover, according to recent studies on luminescent small molecules, such rigid structures in the crystalline state are capable of restricting vibrational/rotational movements during the electronic transitions and thus alter their optical properties (Li et al., “Fluorescence of Nonaromatic Organic Systems and Room Temperature Phosphorescence of Organic Luminogens: The Intrinsic Principle and Recent Progress,” Small 14(38):1801560 (2018); Mei et al., “Aggregation-Induced Emission: Together We Shine, United We Soar!,” Chem. Rev 115(21):11718-11940 (2015); and Mei et al., “Aggregation-Induced Emission: The Whole Is More Brilliant Than the Parts,” Advanced Materials 26(31):5429-5479 (2014), which are hereby incorporated by reference in their entirety). Therefore, it is anticipated that the restriction of the rotation/vibration due to the close packing of amino acids (Gopikrishna et al., “Monosubstituted Dibenzofulvene-Based Luminogens: Aggregation-Induced Emission Enhancement and Dual-State Emission,” The Journal of Physical Chemistry C 120(46):26556-26568 (2016) and Dong et al., “Switching the Light Emission of (4-biphenylyl) Phenyldibenzofulvene by Morphological Modulation: Crystallization-Induced Emission Enhancement,” Chemical Communications (1):40-42 (2007), which are hereby incorporated by reference in their entirety), the stronger intramolecular n-π and π-π coupling interactions (Li et al., “Fluorescence of Nonaromatic Organic Systems and Room Temperature Phosphorescence of Organic Luminogens: The Intrinsic Principle and Recent Progress,” Small 14(38):1801560 (2018), which is hereby incorporated by reference in its entirety), and the hydrogen bonding network in the crystalline state (compared to the solution state) may all account for the fluorescence emission of the amino acids (Mei et al., “Aggregation-Induced Emission: Together We Shine, United We Soar!,” Chem. Rev 115(21):11718-11940 (2015); Mei et al., “Aggregation-Induced Emission: The Whole Is More Brilliant Than the Parts,” Advanced Materials 26(31):5429-5479 (2014); Zhang et al., “Intramolecular Vibrations in Low-Frequency Normal Modes of Amino Acids: L-Alanine in the Neat Solid State,” Acta Chemica Scandinavica 119(12):3008-3022 (2015); Dong et al., “Piezochromic Luminescence Based On the Molecular Aggregation of 9, 10-Bis ((E)-2-(pyrid-2-yl) vinyl) Anthracene,” Angewandte Chemie International Edition 51(43):10782-10785 (2012); and Han et al., “A Diethylaminophenol Functionalized Schiff Base: Crystallization-Induced Emission-Enhancement, Switchable Fluorescence and Application for Security Printing and Data Storage,” Journal of Materials Chemistry C 3(28):7446-7454 (2015), which are hereby incorporated by reference in their entirety). The same phenomenon, interactions conducted by carbonyl and amino moieties, has been suggested to explain the fluorescence properties of poly(amido amine) (PAMAM) in its aggregated state (Wang et al., “Aggregation-Induced Emission of Non-Conjugated Poly (Amido Amine) S: Discovering, Luminescent Mechanism Understanding and Bioapplication,” Chinese Journal of Polymer Science 33(5):680-687 (2015), which is hereby incorporated by reference in its entirety).

### Example 11—Macrostructural Differences in Amino Acid Crystals

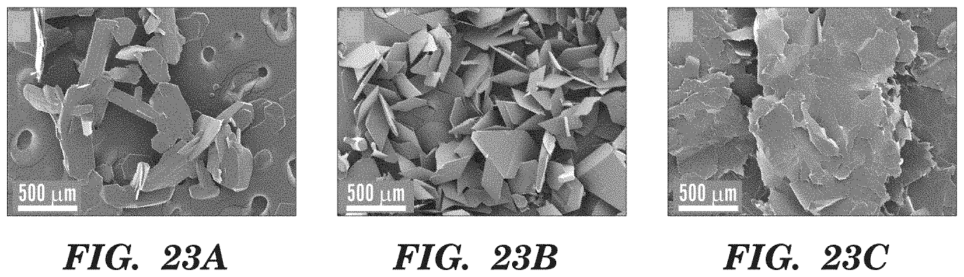

It has recently been shown that the optical properties of organic crystals are intimately linked to their crystal macrostructure and the relative spatial arrangement of those molecules across many length scales within the crystal (Potticary et al. “Nanostructural Origin of Blue Fluorescence in the Mineral Karpatite,” Scientific Reports 7(1):9867 (2017), which is hereby incorporated by reference in its entirety). This phenomenon may explain the different fluorescence emission intensities that were observed for the amino acid crystals depending on their molecular structure (FIGS. 9A-9B, 11-15). Thus, the macrostructural differences in the amino acid crystals were investigated using scanning electron microscopy (SEM) to better understand how it may affect their optical behavior (FIG. 10, FIG. 23A-23C). The SEM images demonstrate differences between the macrostructures of the amino acid crystals. However, no specific relationship between these macrostructures and the amino acids' fluorescence emission intensity was observed. This study sheds light on a general strategy to induce the fluorescence of nonaromatic compounds by taking advantage of the readily available non-covalent interactions in the assembled crystalline form.

### Discussion of Examples 8-11

Due to the application of long lived luminescent solid organic materials in electroluminescent devices, sensors, and cell imaging, there has been a resurgent interest in the past few years towards the development of new organic molecules with room temperature fluorescence in the solid state (Mei et al., “Aggregation-Induced Emission: The Whole Is More Brilliant Than the Parts,” Advanced Materials 26(31):5429-5479 (2014) and Baroncini et al., “Rigidification Or Interaction-Induced Phosphorescence of Organic Molecules,” Chemical Communications 53(13):2081-2093 (2017), which are hereby incorporated by reference in their entirety). Examples 8-11 demonstrate that pure crystals of L-histidine, L-glutamine, L-isoleucine, L-asparagine, L-valine, L-threonine, and L-methionine amino acids are fluorescent at room temperature, while none of these molecules are fluorescent in solution. Crystal structure, an emergent property, is not simply related to molecular structure (Desiraju, G. R., “Crystal Engineering: From Molecule To Crystal,” Journal of the American Chemical Society 135(27):9952-9967 (2013), which is hereby incorporated by reference in its entirety). The results described herein confirm this statement and anticipate that the restriction of intramolecular motion and electronic interactions among electron-rich groups in amino acids favored by their close proximity in the crystalline state are the most important factors for observing fluorescent amino acid crystals. However, applicant notes that a conformation may also be responsible for the differences observed in the fluorescence emission intensity of these aromatic and nonaromatic amino acids. With the understanding that active intramolecular motion can effectively dissipate exciton energy, while restricted intramolecular motions can activate radiative transitions, numerous opportunities can be explored. Indeed, the principle of crystallization-induced emission may trigger new developments in an array of fields, ranging from bioimaging, chemosensing, optoelectronics, and stimuli-responsive systems (Mei et al., “Aggregation-Induced Emission: Together We Shine, United We Soar!,” Chem. Rev 115(21):11718-11940 (2015); Ravanfar et al., “Controlling the Release From Enzyme-Responsive Microcapsules With a Smart Natural Shell,” ACS Applied Materials & Interfaces 10(6):6046-6053 (2018); Ravanfar et al., “Thermoresponsive, Water-Dispersible Microcapsules With a Lipid-Polysaccharide Shell To Protect Heat-Sensitive Colorants,” Food Hydrocolloids 81:419-428 (2018); and Ravanfar et al., “Preservation of Anthocyanins in Solid Lipid Nanoparticles: Optimization of a Microemulsion Dilution Method Using the Placket-Burman and Box-Behnken Designs,” Food Chemistry 199:573-580 (2016), which are hereby incorporated by reference in their entirety).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An encapsulated product comprising:
(i) one or more amino acids, wherein the one or more amino acids are in the form of a crystal with one or more hydrophobic domains and
(ii) one or more hydrophobic agents entrapped within the hydrophobic domains of the crystal of the one or more amino acids, said crystal having a hydrophilic exterior.

2. The encapsulated product of claim 1, wherein the one or more amino acids are aromatic, non-aromatic, or combinations thereof.

3. The encapsulated product of claim 2, wherein the aromatic amino acids are selected from the group consisting of histidine, phenylalanine, tyrosine, and tryptophan.

4. The encapsulated product of claim 2, wherein the non-aromatic amino acids are selected from the group consisting of glutamine, isoleucine, asparagine, valine, threonine, and methionine.

5. The encapsulated product of claim 1, wherein the one or more amino acids are L-amino acids, D-amino acids, or combinations thereof.

6. The encapsulated product of claim 1, wherein the one or more amino acids is L-histidine.

7. The encapsulated product of claim 1, wherein the one or more amino acids are monomers, dimers, trimers, or combinations thereof.

8. The encapsulated product of claim 1, wherein the one or more hydrophobic agents are selected from the group consisting of vitamins, carotenoids, antioxidants, drugs, imaging agents, and combinations thereof.

9. The encapsulated product of claim 8, wherein the one or more hydrophobic agents is a carotenoid selected from the group consisting of β-carotene, alpha-carotene, lycopene, lutein, zeaxanthin, beta cryptoxanthin, and combinations thereof.

10. The encapsulated product of claim 8, wherein the one or more hydrophobic agents is a drug selected from the group consisting of anticancer agents and antimicrobial agents.

11. The encapsulated product of claim 10, wherein the one or more hydrophobic agents is an anticancer agent selected from the group consisting of doxorubicin HCl (Dox), paclitaxel (PTX), 5-fluorouracil, camptothecin, cisplatin, metronidazole, melphalan, docetaxel, and combinations thereof.

12. The encapsulated product of claim 10, wherein the one or more hydrophobic agents is an antimicrobial agent selected from the group consisting of doxycycline, cephalexin, gentamycin, kanamycin, rifamycins, novobiocin, and combinations thereof.

13. The encapsulated product of claim 8, wherein the one or more hydrophobic agents is an imaging agent selected from the group consisting of Nile red, pyrene, anthracene, and combinations thereof.

14. The encapsulated product of any one of claims 1-13, wherein the hydrophilic exterior is covalently modified to comprise a targeting agent.

15. The encapsulated product of claim 14, wherein targeting agent is a polymer selected from the group consisting of hyaluronic acid (HA), polysialic acid (PSA), polyethylene glycol (PEG), and combinations thereof.

16. The encapsulated product of claim 14, wherein the crystal is fluorescent.

17. A pharmaceutical or cosmetic composition comprising a pharmaceutically or cosmetically acceptable carrier and the encapsulated product according to one of claims 1-16.

18. The pharmaceutical or cosmetic composition of claim 17, wherein the composition is suitable for administration orally, topically, transdermally, parenterally, intradermally, intrapulmonary, intramuscularly, intraperitoneally, intravenously, subcutaneously, or by intranasal instillation, by intracavitary or intravesical instillation, intraocularly, intraarterialy, intralesionally, or by application to mucous membranes.

19. The pharmaceutical or cosmetic composition of claim 17, wherein the hydrophobic agent is present at a concentration of about 0.1-65%.

20. A method of therapeutically treating a subject with one or more hydrophobic agents, said method comprising:
selecting a subject in need of therapeutic treatment and
administering the encapsulated product according to claims 1-12 or 14-16 or the pharmaceutical or cosmetic composition according to claims 17-19 to the selected subject.

21. A method of in vitro imaging, said method comprising:
selecting an in vitro cell culture system;
contacting the in vitro cell culture system with the encapsulated product according to claims 1-16 or a pharmaceutical or cosmetic composition according to claims 17-19; and
imaging the contacted cell culture system.

22. The method of claim 20 or claim 21, wherein said administering or contacting is repeated.

23. The method of claim 22, wherein said administering or contacting is carried out daily, weekly, or monthly.

24. The method of claim 20, wherein the subject is in need of treatment for cancer.

25. The method of claim 20, wherein the subject is in need of treatment for a vitamin deficiency.

26. The method of claim 20, wherein the subject is in need of treatment for disease selected from the group consisting of a dermatological disorder, dermatological disease, or dermatological imperfection.

27. The method of claim 20, wherein the subject is in need of treatment for an infectious disease.

28. The method of claim 20, wherein the subject is a mammalian subject.

29. The method of claim 20, wherein the subject is a human subject.

30. The method of claim 21, wherein the in vitro cell culture system comprises primary cells.

31. The method of claim 21, wherein the in vitro cell culture system comprises a cell line.

32. The method of claim 21, wherein the in vitro cell culture system comprises mammalian cells.

33. The method of claim 32, wherein the mammalian cells are human cells.

34. The method of claim 21, wherein said imaging is carried out by confocal microscopy.

35. A method of preparing an encapsulated product comprising entrapped hydrophobic agents, said method comprising:
mixing one or more hydrophobic agents with one or more amino acids to produce a mixture and
forming crystals of the one or more amino acids entrapping the one or more hydrophobic agents, wherein the crystals have a hydrophilic exterior.

36. The method of claim 35, wherein said mixing is carried out in an aqueous solution.

37. The method of claim 35, wherein said mixing and incubating steps are carried out at a temperature of 0° C. to 60° C.

38. The method of claim 35, wherein the one or more amino acids are aromatic, non-aromatic, or combinations thereof.

39. The method of claim 38, wherein the aromatic amino acids are selected from the group consisting of histidine, phenylalanine, tyrosine, and tryptophan.

40. The method of claim 38, wherein the non-aromatic amino acids are selected from the group consisting of glutamine, isoleucine, asparagine, valine, threonine, and methionine.

41. The method of claim 35, wherein the one or more amino acids are L-amino acids, D-amino acids, or combinations thereof.

42. The method of claim 35, wherein the one or more amino acids is L-histidine.

43. The method of claim 35, wherein the one or more amino acids are monomers, dimers, trimers, or combinations thereof.

44. The method of claim 35, wherein the one or more hydrophobic agents are selected from the group consisting of vitamins, carotenoids, antioxidants, drugs, imaging agents, and combinations thereof.

45. The method of claim 44, wherein the one or more hydrophobic agents is a carotenoid selected from the group consisting of β-carotene, alpha-carotene, lycopene, lutein, zeaxanthin, beta cryptoxanthin, and combinations thereof.

46. The method of claim 44, wherein the one or more hydrophobic agents is a drug selected from the group consisting of chemotherapeutic agents and antibiotic agents.

47. The method of claim 46, wherein the one or more hydrophobic agents is a chemotherapeutic agent selected from the group consisting of doxorubicin HCl (Dox), paclitaxel (PTX), 5-fluorouracil, camptothecin, cisplatin, metronidazole, melphalan, docetaxel, and combinations thereof.

48. The method of claim 46, wherein the one or more hydrophobic agents is an antibiotic agent selected from the group consisting of doxycycline, cephalexin, gentamycin, kanamycin, rifamycins, novobiocin, and combinations thereof.

49. The method of claim 46, wherein the one or more hydrophobic agents is an imaging agent selected from the group consisting of Nile red, pyrene, anthracene, and combinations thereof.

50. The method of claim 35, wherein the mixture further comprises an antisolvent.

51. The method of claim 50, wherein the antisolvent is selected from the group consisting of ethanol, methanol, Tetrahydrofuran, acetone, and combinations thereof.

52. The method of claim 35 further comprising:
washing the crystals to remove unentrapped hydrophobic agents and
modifying the washed crystals' surfaces to include a targeting agent.

53. The method of claim 52, wherein the targeting agent is a polymer selected from the group consisting of hyaluronic acid (HA), polysialic acid (PSA), polyethylene glycol (PEG), and combinations thereof.

54. The method of claim 53, wherein the targeting agent is hyaluronic acid.
